(19) United States
(12) Patent Application Publication
(10) Pub. No.: US 2003/0008914 A1
(43) Pub. Date: Jan. 9, 2003

(54) PHARMACEUTICAL FORMULATIONS USEFUL TO TREAT INFLAMMATORY AND IMMUNE DISORDERS

(76) Inventors: C. GRACE YEH, Marlboro, MA (US); GORDON J. DOW, Santa Rosa, CA (US); ROBERT W. LATHROP, Novato, CA (US); MUKAND S. CHORGHADE, Wellesley, MA (US); ALLA VERKATA RAMA RAO, Hyderabad (IN)

Correspondence Address: DIKE BRONSTEIN ROBERTS & CUSHAMN, 130 WATER STREET, BOSTON, MA, 02109

(21) Appl. No.: 09/419,354

(22) Filed: Oct. 15, 1999

Related U.S. Application Data
(60) Provisional application No. 60/172,213, filed on Oct. 16, 1998.

(51) Int. Cl. A61K 31/47; A01N 43/08

(52) U.S. Cl. 514/471

(57) ABSTRACT

A pharmaceutical formulation is provided for the treatment of inflammatory and/or immune disorders, particularly those mediated by platelet activating factor (“PAF”) or a product of 5-lipoxygenase. The formulation, in one embodiment, is an essentially anhydrous ointment containing an active agent selected from the group consisting of 2,5-diaryl tetrahydrofurans, 2,5-diaryl tetrahydrothiophenes, 2,4-diaryl tetrahydrofurans, 2,4-diaryl tetrahydrothiophenes, 1,3-diaryl cyclopentanes, 2,4-diaryl pyrrolidines, and 2,5-diaryl pyrrolidines, and an enhancer composition containing one or more C₃₋₁₈ esters such as diethyl succinate, propylene carbonate, diisopropyl adipate and glyceryl triacetate. In another embodiment, the formulation is a cream, gel, lotion, oil, or the like, containing the: active agent in crystalline form. The invention also encompasses the novel crystalline form of the active agent, and methods for using the formulations to treat individuals with inflammatory and/or immune disorders. Also encompassed is use of isopropyl alcohol (IPA) to enhance stability of the active agent and pharmaceutical formulations.

### CROSS-REFERENCE TO RELATED APPLICATION

The present application claims the benefit of U.S. provisional application originally having application Ser. No. 09/173,903, filed Oct. 16, 1998, incorporated herein by reference in its entirety.

### TECHNICAL FIELD

The present invention relates generally to drug delivery, and more particularly relates to pharmaceutical formulations and methods for treating inflammatory and immune disorders using the novel formulations.

### BACKGROUND

Allergy, asthma, autoimmune disorders and tissue injury are known to induce the release of lipid mediators, leukotrienes generated by the 5-lipoxygenase (“5-LO”) pathway and platelet activating factor (“PAF”; 1-O-alkyl-2-acetyl-sn-glycerol-3-phosphoryl choline) from leukocytes. Leukotrienes and PAF trigger the major symptoms of inflammatory diseases: bronchoconstriction, cellular infiltration, swelling, congestion and pain.

Recent efforts in identifying and developing effective agents to treat inflammatory and immune disorders have led to the synthesis of a family of important compounds, described in detail in U.S. Pat. No. 5,434,151 for “Compounds and Methods for the Treatment of Disorders Mediated by Platelet Activating Factor or Products of 5-Lipoxygenase,” inventors Cai et al., assigned to CytoMed, Inc. (Cambridge, Mass.). Those compounds reduce damage arising from an inflammatory or immune response by acting as receptor antagonists of platelet activating factor or by inhibiting the activity of 5-lipoxygenase, or both. As described in detail in the aforementioned patent, the compounds are 2,5-diaryl tetrahydrothiophenes, tetrahydrofurans, and pyrrolidines, 1,3-diaryl cyclopentanes, and 2,4-diaryl tetrahydrothiophenes, tetrahydrofurans and pyrrolidines. An exemplary compound is (±) trans-2-[5-(N′-methyl-N′-hydroxyureidylmethyl)-3-methoxy-4-p-chlorophenylthioethoxyphenyl]-5-(3,4,5-trimethoxyphenyl)tetrahydrofuran, sometimes referred to herein as “CMI-392” and shown in the following structural formula:

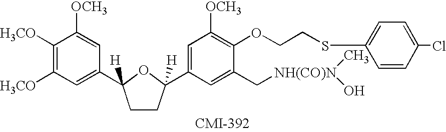

CMI-392, a compound that, uniquely, is both a 5-LO inhibitor and a PAF receptor antagonist, has proved to be an extremely effective agent for treating inflammatory and immune disorders, as have the other compounds set forth in the Cai et al. patent. The compounds have been found to be particularly useful in treating psoriasis and atopic dermatitis, both chronic inflammatory skin disorders affecting millions of people. A number of pharmaceutical compositions containing these drugs have been proposed and prepared, However, there remains a need for a formulation which allows for enhanced penetration of the drug into the skin or mucosal tissue, is stable under typical storage conditions for at least several months, and can be prepared as a cream or other composition containing water, alcohol, or other hydrophilic components.

Prior to the present invention, the only known process for synthesizing and purifying CMI-392—as disclosed in U.S. Pat. No. 5,434,151 to Cai et al.—resulted in a waxy, low melting point solid that proved to be difficult to work with and sensitive to heat and moisture. Temperatures below about 4° C. are required for storage, and exposure to ambient temperature, light, and the like, results in degradation to a discolored melt. The instability of the drug has limited its potential for manufacturing and scale-up; in addition, the waxy nature of the material has precluded purification on a large scale. Preparation of alcohol-containing or water-containing gels, lotions and creams, has not, until now, been possible, as the original form of the drug was unstable in water and other protic solvents. Furthermore, the fact that many skin permeation enhancers are hydrophilic compounds, e.g., lower alkanols or the like, has prevented the combination of such compounds with CMI-392 in a topical formulation.

The inventors herein have now discovered a new pharmaceutical formulation of CMI-392 and/or other structurally similar antiinflammatory agents as described in the Cai et al. patent, comprising an ointment that is stable to heat, light and moisture, in which the active agent is itself stabilized, and which provides for efficient and effective penetration of the drug into the epidermal and dermal skin layers. In addition, a new, crystalline form of CMI-392 has been prepared that is stable in a variety of different environments, i.e., is far more stable to heat, light and moisture than the original form of the compound. The crystalline form of the drug allows for the preparation of a number of different types of compositions, including ointments, creams, oils, solutions and gels, and the incorporation therein of a variety of components which can serve as vehicles and stabilization enhancers.

### SUMMARY OF THE INVENTION

Accordingly, it is a primary object of the invention to address the above-mentioned need in the art by providing a pharmaceutical formulation for treating inflammatory and/or immune disorders.

It is also an object of the invention to provide such a formulation for topical administration, i.e., for administration to the skin, mucosal tissue or eye.

It is another object of the invention to provide a novel pharmaceutical formulation comprising an ointment containing CMI-392 or a structurally similar diaryl-substituted cyclic or heterocyclic compound, in combination with an enhancer composition effective to increase formulation stability and enhance the penetration of the drug into the skin.

It is an additional object of the invention to provide CMI-392 or a structurally similar diaryl-substituted cyclic or heterocyclic compound in a novel crystalline form.

It is a further object of the invention to provide a pharmaceutical formulation containing one or more suitable topical vehicles and/or enhancers, and CMI-392 or a structurally similar diaryl-substituted cyclic or heterocyclic compound, in novel crystalline form.

It is still a further object of the invention to provide a method for treating an individual with an inflammatory or immune disorder by administering a pharmaceutical formulation as disclosed and claimed herein.

Additional objects, advantages and novel features of the invention will be set forth in part in the description which follows, and in part will become apparent to those skilled in the art upon examination of the following, or may be learned by practice of the invention.

In one aspect of the invention, then, a pharmaceutical formulation is provided in the form of an ointment containing an active agent selected from the group consisting of 2,5-diaryl tetrahydrofurans, 2,5-diaryl tetrahydrothiophenes, 2,4-diaryl tetrahydrofurans, 2,4-diaryl tetrahydrothiophenes, 1,3-diaryl cyclopentanes, 2,4-diaryl pyrrolidines, and 2,5-diaryl pyrrolidines. Preferred active agents are 2,5-diaryl tetrahydrofurans (exemplary compounds are shown in Formula (I), below, wherein X is O), and a particularly preferred active agent is CMI-392. The ointment also contains an enhancer composition comprising an ester, typically an ester that is liquid at room temperature and has a molecular weight of less than about 250. It has been found that such ester-containing enhancer compositions stabilize CMI-392 and other analogous active agents as disclosed herein, and also provide for increased penetration of the drug into and/or through the skin, mucosal tissue or eye.

Oil-based formulations of the invention also are preferred, particularly oil formulations that contain an alcohol component, especially a branched C₁₋₁₂ alcohol such as isopropyl alcohol.

In another aspect of the invention, a novel crystalline form of the active agent is provided. The new form of the drug, which may be CMI-392 or an alternative active agent as described herein, has enhanced stability and increased compatibility with water, alcohol-containing solutions, and other protic materials. Thus, this form of the active agent enables preparation of a variety of pharmaceutical formulations such as gels, creams, lotions, solutions, oils, suppositories and the like.

Methods of using the novel formulations to treat individuals with inflammatory and/or immune disorders are also provided. As the active agents herein are PAF receptor antagonists and 5-lipoxygenase inhibitors, the methods of use involve treatment of disorders mediated by PUFF or leukotrienes. Leukotrienes, as is known in the art, are produced by the oxidation of arachidonic acid by lipoxygenases, including 5-lipoxygenase, and play a major role in inflammatory and allergic responses.

### BRIEF DESCRIPTION OF THE DRAWINGS

FIGS. 1a-1c schematically illustrate synthesis of CMI-392 from 3,4,5-trimethoxyacetophenone, as described in Examples 1 and 4.

FIGS. 2a-2c schematically illustrate synthesis of CMI-392 in crystalline form from 3,4,5-trimethoxy benzoic acid, as described in Example 2.

FIGS. 3a-3c schematically illustrate an alternative method for synthesizing crystalline CMI-392 using acetovanillone as a starting material, as described in Example 3.

FIGS. 4a-4c schematically illustrate an alternative method for synthesizing crystalline CMI-392 using acetyl salicylic acid (aspirin) as a starting material.

FIG. 5 is a DSC of crystalline CMI-392, taken over a temperature range of 25 to 350° C. (heating rate 10° C./min.).

FIGS. 6a-6b graphically depict results of Example 14, which follows.

FIG. 7 graphically depicts results of Example 16 which follows.

### DETAILED DESCRIPTION OF THE INVENTION

Definitions:

Before the present formulations, compounds and methods are disclosed and described in detail, it is to be understood that this invention is not limited to specific formulations components, methods of manufacture, or the like, as such may vary. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise. Thus, for example, reference to “an active agent” includes mixtures of active agents, reference to “a vehicle” includes mixtures of two or more vehicles, and the like.

The term “topical administration” is used in its conventional sense to mean delivery of a topical drug or pharmacologically active agent to the skin or mucosa, as in, for example, the treatment of various skin disorders. In general, when the term “skin” is used herein, as in administration of a topical formulation to “the skin,” it is to be understood that administration to mucosal tissue is intended as a possibility as well.

“Topical” delivery is additionally intended to include administration to the eye. Colonic delivery, e.s., by way of an enema solution or suppository is also encompassed herein.

The terms “active agent,” “drug” and “pharmacologically active agent” are used interchangeably herein to refer to a chemical material or compound that is suitable for topical administration and induces the desired physiological effect. The terms include derivatives, analogs and prodrugs of such compounds, as well as pharmaceutically acceptable salts, esters, amides, and the like.

By the term “effective” amount of a drug is meant a nontoxic but sufficient amount of a compound, to provide the desired effect and performance at a reasonable benefit/risk ratio attending any medical treatment.

The terms “vehicle” and “carrier” as used herein refer to pharmaceutically acceptable vehicles and carriers. The terms “topical vehicle” or “topical carrier” as used herein specifically refer to a vehicle suitable for topical application of a drug.

“Penetration enhancement” or “permeation enhancement” as used herein relates to an increase in the permeability of the skin or mucosal tissue to the selected pharmacologically active agent, which in turn gives rise to an increase in the rate at which the drug permeates into and/or through the skin or mucosal tissue.

The term “PAF receptor antagonist” refers to a compound that binds to a PAF receptor with a binding constant of 30:M or lower.

The term “5-lipoxygenase inhibitor” refers to a compound that inhibits the enzyme at 30 μM or lower, as may be evaluated in a broken cell system.

The term “enantiomerically enriched composition” refers to a composition that includes at least 95% by weight of a single enantiomer of the compound.

With respect to the description of chemical structures and substituents contained therein, the following definitions are applicable:

The term “alkyl” as used herein, unless otherwise specified, refers to a saturated straight chain, branched or cyclic hydrocarbon group of 1 to 10 carbon atoms, such as methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The term “lower alkyl” intends an alkyl group of one to six carbon atoms, and includes, for example, methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl.

The term “alkenyl” as used herein, unless otherwise specified, refers to a branched, unbranched or cyclic (in the case of C₅ and C₆) hydrocarbon group of 2 to 10 carbon atoms containing at least one double bond, such as ethenyl, vinyl, allyl, octenyl, decenyl, and the like. The term “lower alkenyl” intends an alkenyl group of two to six carbon atoms, and specifically includes vinyl and allyl.

The term “alkynyl” as used herein, unless otherwise specified, refers to a branched or unbranched hydrocarbon group of 2 to 10 carbon atoms containing at least one triple bond, such as acetylenyl, ethynyl, n-propynyl, isopropynyl, n-butynyl, isobutynyl, t-butynyl, octynyl, decynyl and the like. The term “lower alkynyl” intends an alkynyl group of two to six carbon atoms, and includes, for example, acetylenyl and propynyl.

The term “lower alkylamino” as used herein, and unless otherwise specified, refers to an amino group that has one or two lower alkyl substituents.

The term “aryl” as used herein, and unless otherwise specified, refers to phenyl or substituted phenyl, wherein the substituent is halo or lower alkyl.

The term “halo” is used in its conventional sense to refer to a chloro, bromo, fluoro or iodo substituent. The terms “haloalkyl,” “haloalkenyl” or “haloalkynyl” (or “halogenated alkyl,” “halogenated alkenyl,” or “halogenated alkynyl”) refers to an alkyl, alkenyl or alkynyl group, respectively, in which at least one of the hydrogen atoms in the group has been replaced with a halogen atom.

The terms “heterocycle” or “heteroaromatic,” as used herein, and unless otherwise specified, refer to an aromatic moiety that includes at least one sulfur, oxygen or nitrogen atom in the aromatic ring. Such moieties include, but are not limited to, pyrryl, furyl, pyridyl, 2,4-thiadiazolyl, pyrimidyl, thienyl, isothiazolyl, imidazolyl, tetrazolyl, pyrazinyl, pyrimidyl, quinolyl, isoquinolyl, benzothienyl, isobenzofuryl, pyrazolyl, indolyl, purinyl, carbazolyl, benzimidazolyl and isoxazolyl.

The term “aralkyl” refers to an aryl group with an alkyl substituent.

The term “alkaryl” refers to an allyl group that has an aryl substituent.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

The Active Agent:

The topical pharmaceutical formulations of the invention contain, as the active agent suitable for treating inflammatory and/or immune disorders, a diaryl-substituted cyclic or heterocyclic compound as described in U.S. Pat. No. 5,434,151 to Cai et al., cited earlier herein. Such compounds are 2,5-diaryl tetrahydrothiophenes, tetrahydrofurans, and pyrrolidines, 1,3-diaryl cyclopentanes, and 2,4-diaryl tetrahydrothiophenes, tetrahydrofurans and pyrrolidines. The compounds have the structural formulae (I), (II) or (III):

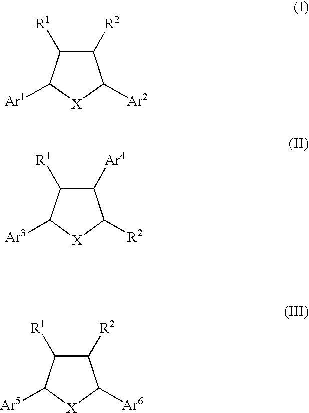

wherein the various substituents are as follows:

R¹ and R² are independently selected from the group consisting of: hydrogen; lower alkyl, e.g., methyl, cyclopropylmethyl, ethyl, isopropyl, butyl, pentyl and hexyl; C₃-C₈ cycloalkyl, e.g., cyclopentyl; halogenated lower alkyl, e.g., trifluoromethyl; halo, particularly fluoro; —COOH; —CONR¹⁶R¹⁷ wherein R¹⁶ and R¹⁷ independently represent lower alkyl or hydrogen; lower alkenyl, e.g., vinyl, allyl, CH₃CH═CH—CH₂—CH₂— and CH₃(CH₂)₃—CH═CH—; lower alkynyl, e.g., acetylenyl; ═O; —OR³; —COOR³; —C(O)R³; —CH₂R³; —CH₂NR³R⁴; and —NR³R⁴, wherein

R³ and R⁴ are independently alkyl, alkenyl, alkynyl, aryl, aralkyl, alkaryl, hydrogen, C₁₋₆ alkoxy-C₁₋₁₀ alkyl, C₁₋₆ alkylthio-C₁₋₁₀ alkyl; and C₁₋₁₀ substituted alkyl (wherein the substituent is independently hydroxyl or carbonyl, located on any one of C₁₋₁₀).

X is O, S, S(O), S(O)₂, CR⁹ or NR¹⁰ wherein R⁹ is independently hydrogen, halogen, lower alkyl, halogenated lower alkyl, lower alkenyl, lower alkynyl, —CONR³R⁴, —C(O)R⁵, —CH₂OR⁵, —CH₂NR⁵R⁵, —CH₂SR⁵, ═O, ═NR⁵, —NR³R⁴, or —OR⁵, and R¹⁰ is —R³, —R⁸, —C(O)N(OR³)R³ or —OR³, where R³ and R⁴ are as defined above, R⁵ is lower alkyl, lower alkenyl, lower alkynyl, hydroxyl, hydrogen, halogenated lower alkyl, halogenated lower alkenyl, halogenated lower alkynyl, aralkyl or aryl, and R⁸ is halogenated alkyl, halogenated lower alkyl, halogenated lower alkenyl, halogenated lower alkynyl, lower alkenyl, lower alkynyl, a-alkyl or aryl.

Ar¹ is

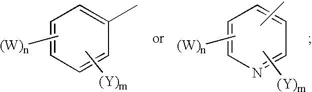

Ar² is

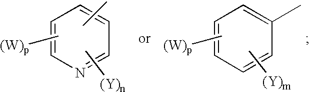

Ar³ and Ar⁴ are independently

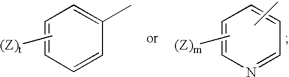

Ar⁵ is

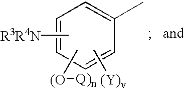

Ar⁶ is

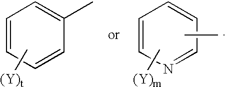

W is independently:

(1) —AN(OM)C(O)NR³R⁴, —ANR³C(O)N(OM)R⁴, —AN(OM)C(O)R⁴, —AC(O)N(OM)R⁴, —N(OM)C(O)NR³R⁴, —NR³C(O)N(OM)R⁴, —N(OM)C(O)R⁴, —C(O)N(OM)R⁴, —OR⁶NR⁵R⁶—(C₅H₄N)R⁶R⁷, —OR⁶N(COR⁵)R⁶—(C₅H₄N)R⁶R⁷, —OR⁶OC(O)N(COR⁵)R⁶—(C₅H₄N)R⁶R⁷, —OR⁶O(CO)N(CO₂R⁶)R⁶(C₅H₄N)R⁶R⁷, —A(C₅H₄N)R⁶R⁷ or —OR⁶N(CO₂R⁵)R⁶—(C₅H₄N)R⁶R⁷;

(2) an amidohydroxyurea of the formula —NR¹⁹C(O)C(R¹⁹)₂N(OM)—C(O)NHR²⁰, —C(O)NR¹⁹C(R¹⁹)₂N(OM)C(O)NHR²⁰, —ANR¹⁹C(O)C(R¹⁹)₂N(OM)—C(O)NHR²⁰, —AC(O)NR¹⁹C(R¹⁹)₂N(OM)C(O)NHR²⁰, —NHC(O)N(OM)C(R¹⁹)₂—C(O)N(R¹⁹)₂ or —NHC(O)N(OM)C(R¹⁹)₂N(R¹⁹)₂C(O)R¹⁹;

(3) an oxalkane of the structure

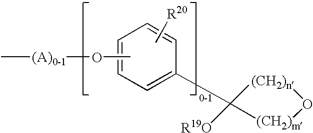

in which m′ and n′ are independently 1-4;

(4) a thioalkane of the structure

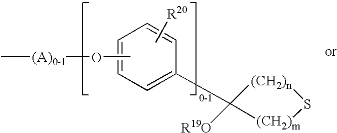

(5) a quinolylmethoxy of the structure

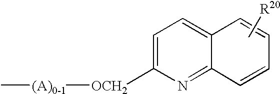

In the aforementioned possibilities for W, the substituents R³, R⁴ and R⁵ are as defined above, R⁶ is lower alkyl, lower alkenyl, lower alkynyl, aralkyl, halogenated lower alkyl, halogenated lower alkenyl, halogenated lower alkynyl or aryl, R⁷ is an organic or inorganic anion, R¹⁹ is hydrogen, lower alkyl or lower alkenyl, R²⁰ is hydrogen, halogen, lower alkoxy or lower alkyl, “M” is hydrogen, a pharmaceutically acceptable cation, or a metabolically cleavable leaving group, and “A” is alkyl, alkenyl, alkynyl, alkaryl, aralkyl, halogenated lower alkyl, halogenated lower alkenyl, halogenated lower alkynyl, C₁₋₁₀ alkyl(oxy)-C₁₋₁₀ allyl, C₁₋₁₀ alkylthio-C₁₋₁₀ alkyl, —NR³C(O)alkyl, —NR³C(O)alkenyl, —NR³C(O)alkynyl, —NR³C(O)(alkyl)oxy(alkyl), —NR³C(O)(alkyl)thio(alkyl), —NR³C(O)N(alkyl), —NR³C(O)N(alkenyl), —NR³C(O)N(alkynyl), —NR³C(O)N(alkyl)oxy(alkyl), —N(R³)C(O)N(alkyl)thio-(alkyl), —NR³C(O₂)alkyl, —NR³C(O₂)alkenyl, —NR³C(O₂)alkynyl, —NR³C(O₂)-(alkyl)oxy(alkyl), —NR³C(O₂)(alkyl)thio(alkyl), —OC(O₂)alkyl, —OC(O₂)alkenyl, —OC(O₂)alkynyl, —OC(O₂)(alkyl)oxy(alkyl), —OC(O₂)(alkyl)thio(alkyl), —NR³C(S)alkyl, —NR³C(S)alkenyl, —NR³C(S)alkynyl, —NR³C(S)(alkyl)oxy(alkyl), —NR³C(S)(alkyl)thio(alkyl), —NR³C(S)N(alkyl), —NR³C(S)N(alkenyl), —NR³C(S)N-(alkynyl), —NR³C(S)N(alkyl)oxy(alkyl), —NR³(S)N(alkyl)thio(alkyl), —NR³C(S)S(alkyl), —NR³C(S)S(alkenyl), —NR³C(S)S(alkynyl), —NR³C(S)S(alkyl)-oxy(alkyl), —NR³C(S)S(alkyl)thio(alkyl), —SC(S)S(alkyl), —SC(S)S(alkenyl), —SC(S)S(alkynyl), —SC(S)S(alkyl)oxy(alkyl) or —SC(S)S(alkyl)thio(alkyl).

Y is independently:

(1) hydrogen;

(2) R¹ through R⁶, R⁸, R¹⁰, —OR³, —OR¹¹, —OR¹², R³S—, R⁵S—, R³SO—, R⁵SO—, R³SO₂—, R⁵SO₂—, CF₃S—, CF₃SO—, —CF₃SO₂, —OCH₂-oxycyclopropyl, —OCH₂C(O)OR³, —OCH₂OR³, —OCH₂C(O)R³, —OCH₂C₃₋₈cycloalkyl, —OCH₂CH(R³)R³, —OCH₂-cyclopropyl, —OCH₂-aryl, —OCH₂CH(OH)CH₂OH, aryl-CH₂—SO2—, (R³)₂CHCH₂SO₂—, —CH₂CH(OH)CH₂OH, CF₃SO₂—, R³R⁴N—, —OCH₂CO₂R³, —NR³COR³, —OCONH₂, —OCONR³R⁴, —CONH₂, —CONR³R⁴, —CR³R³R⁴, —SO₂NR³R⁴, —SONR³R⁴, —CH₃OCH₂NR³R⁶, —SNR³R⁴, —CO₂R³, —NR³R⁴SO₂R³, —NR³R⁴SOR³, —COR³, —CONR³, —CN, —NR⁵CONR³R⁴, —CH₂NR⁵CONR³R⁴, —R⁶NR³R⁴, —OR⁶NR³R⁴, —O(O)CR⁵, —O(O)CNR³R⁴,

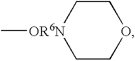

—SR⁶NR³R⁴, —S(O)R⁶NR³R⁴, —SO₂R⁶NR³R⁴,

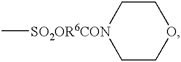

—SR⁶OH, —S(O)R⁶OH, —SO₂R⁶OH, —OR⁶OC(O)N(CO₂R⁶)R⁶, —O-alkyl-N-aryl-C(O)-heterocycle,

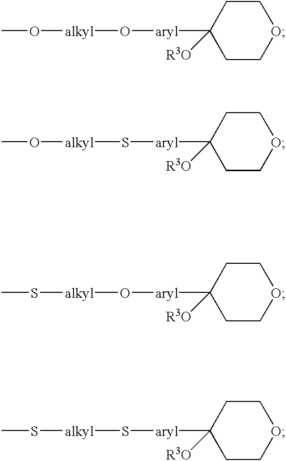

(3) a heterocycle, including but not limited to, pyrryl, furyl, pyridyl; 1,2,4-thiadiazolyl; pyrimidyl, thienyl, isothiazolyl, imidazolyl, tetrazolyl, pyrazinyl, pyrimidyl, quinolyl, isoquinolyl, benzothienyl, isobenzofuryl, pyrazolyl, indolyl, purinyl, carbazolyl, benzimidazolyl and isoxazolyl, optionally substituted with a group described in preceding paragraph, (2);

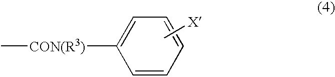

wherein X′ is halo such as F, Cl, Br and I; —C(O)aryl; CF₃; —OR³; —OC(O)NH₂; —CR³R³R⁴; —C(O)R³; —CH₂OR³; —CH₂CO₂R³; —CH₂OC(O)R³; R³CH(R³)CH₂SO₃—; —NHCH₂COOR³; —NR³R³R⁴R⁷⁺; —NR³SO₂R³; —COR³; —NO₂; —CN;

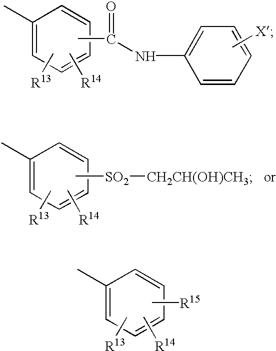

wherein R¹³, R¹⁴ and R¹⁵ independently represent:

BO— wherein B is —CH₂-oxacyclopropyl, —CH₂OR³, —CH₂C(O)R³, —CH₂CH(R³)R³, —CH₂₋aryl, —CH₂CH(OH)—CH₂OH; R³C(R³)₂CH₂SO₂; or R³—R¹⁴ or R¹⁴—R¹⁵ are joined together to form a bridge such as —OCHR²CHR²—S(O)ₙ, wherein n is 0 to 3.

In the aforementioned possibilities for Y:

R¹ through R⁸ and R¹⁰ are as defined above;

R¹¹ is phenyl-S(O)_{g}-lower alkyl-; (R³O)_{d}-phenyl-S(O)_{g}-lower alkyl-; (R³R³N)_{d}-phenyl-S(O)_{g}-lower alkyl-; (CN)_{d}-phenyl-S(O)_{g}-lower alkyl-; (halo)_{d}-phenyl-S(O)_{g}-lower alkyl-; (R³COO)_{d}-phenyl-S(O)_{g}-lower alkyl-; (R³OCO)_{d}-phenyl-S(O)_{g}-lower alkyl-; (R³CO)_{d}-phenyl-S(O)_{g}-lover alkyl-; phenyl-O-lower alkyl-; (R³O)_{d}-phenyl-O-lower alkyl-; (CN)_{d}-phenyl-O-lower alkyl-; (halo)_{d}-phenyl-O-lower alkyl-; (R³COO)_{d}-phenyl-O-lower alkyl-; (R³OCO)_{d}-phenyl-O-lower alkyl-; or (R³CO)_{d}-phenyl-O-lower alkyl- where d is 1, 3, 4 or 5; and g is (0, 1, or 2;and

R¹² is alkyl; substituted alkyl wherein the substituent is selected from the group consisting of hydroxyl- and amino; -lower alkyl-O—R¹⁸, wherein R¹⁸ is —PO₂(OH)⁻M⁺ or —PO₃(M⁺)₂ wherein M⁺ is a pharmaceutically acceptable cation; —C(O)(CH₂)₂CO₂⁻M⁺ or —SO₃⁻M⁺; lower alkylcarbonyl-lower alkyl; -lower alkylamino-lower alkyl; N,N-disubstituted amino lower alkyl-, wherein the substituents each independently represent lower alkyl; pyridyl-lower alkyl; imidazolyl-lower alkyl; imidazolyl-Y-lower alkyl wherein Y is thio or amino; morpholinyl-lower alkyl; pyrrolidinyl-lower alkyl; thiazolinyl-lower alkyl, piperidinyl-lower alkyl; morpholinyl-lower hydroxyalkyl; N-pyrryl; piperazinyl-lower alkyl; N-substituted piperazinyl-lower alkyl, wherein the substituent is lower alkyl; triazolyl-lower alkyl; tetrazolyl-lower alkyl; tetrazolylamino-lower alkyl; or thiazolyl-lower alkyl.

Q is selected from the group consisting of substituted C₁ to C₁₂ alkyl wherein the substituent is selected from the group consisting of hydroxy and amino, alkylcarbonylalkyl, alkyl-, lower alkyl S(O)ₘ-lower alkyl in which m is 1 or 2; imidazolyl lower alkyl, morpholinyl lower alkyl, thiazolinyl lower alkyl, piperidinyl lower alkyl, imidazolylcarbonyl, morpholinyl carbonyl, amorpholinyl (lower alkyl) aminocarbonyl, N-pyrrylpyridinyl-lower alkyl; pyridylthio-lower alkyl; morpholinyl-lower alkyl; hydroxyphenylthio-lower alkyl; cyanophenylthio-lower alkyl; imidazolylthio-lower alkyl; triazolylthio-lower alkyl; triazolylphenylthio-lower alkyl; tetrazolylthio-lower alkyl; tetrazolylphenylthio-lower alkyl; aminophenylthio-lower alkyl; N,N-di-substituted aminophenylthio-lower alkyl wherein the amine substituents each independently represent lower alkyl; amidinophenylthio-lower alkyl, phenylsulfinyl-lower alkyl; or phenylsulfonyl-lower alkyl; -lower alkyl-O—R¹⁸, wherein R¹⁸ is —PO₂(OH)⁻M⁺ or —PO₃(M⁺)₂, wherein M⁺ is a pharmaceutically acceptable cation; —C(O)(CH₂)₂CO₂⁻M⁺, or —SO₃⁻M⁺; -lower alkylcarbonyl-lower alkyl; -carboxy lower alkyl, -lower alkylamino-lower alkyl; N,N-di-substituted amino lower alkyl, wherein the amine substituents each independently represent lower alkyl; pyridyl-lower alkyl; imidazolyl-lower alkyl; imidazolyl-Y-lower alkyl wherein Y is thio or amino; morpholinyl-lower alkyl; pyrrolidinyl-lower alkyl; thiazolinyl-lower alkyl, piperidinyl-lower alkyl; morpholinyl-lower hydroxyalkyl; N-pyrryl; piperazinyl-lower alkyl; N-substituted piperazinyl-lower alkyl, wherein the amine substituent is lower alkyl; triazolyl-lower alkyl; tetrazolyl-lower alkyl; tetrazolylamino-lower alkyl; or thiazolyl-lower alkyl.

In Formula (I) compounds, m is 1, 2 or 3, n is 1 or 2, p is 0 or 1; in Formula (II) compounds, m is as just defined, and t is 1, 2, 3 or 4. In Formula (E) compounds, m and n are as just defined, and v is 0, 1 or 2.

Examples of specific compounds of Formula (I), (II) and (III) are set forth in U.S. Pat. No. 5,434,151 to Cai et al., cited above. Preferred compounds within these formulae have the structure (IV)

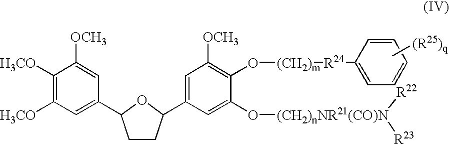

in which n is 0 or 1, m is 2 or 3, q is 1, 2, 3 or 4, R²¹ is H or OH, R²² is H or OH, R²³ is lower alkyl, preferably C₁ to C₄ alkyl, R²⁴ is S or SO₂, and R²⁵ is lower alkyl, lower alkoxy or halide.

A particularly preferred compound is (±) trans-2-[5-(N′-methyl-N′-hydroxyureidyl-methyl)-3-methoxy-4-p-chlorophenylthioethoxyphenyl]-5-(3,4,5-trimethoxyphenyl)tetrahydrofuran, CMI-392:

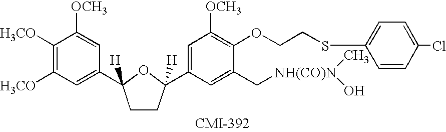

Additional particularly preferred compounds, structural variants of CMI-392, are (±) trans-2-[5-(N′-methyl-N′-hydroxyureidylmethyl)-3-methoxy-4-p-chlorophenylthiopropoxyphenyl]-5-(3,4,5-trimethoxyphenyl)tetrahydrofuran, (±) trans-2-[5-(N′-methyl-N′-hydroxyureidylmethyl)-3-methoxy-4-p-fluorophenylthioethoxyphenyl]-5-(3,4,5-trimethoxyphenyl)tetrahydrofuran, and (±) trans-2-[5-(N′-methyl-N′-hydroxyureidylmethyl)-3-methoxy-4-p-fluorophenylthiopropoxy-phenyl]-5-(3,4,5-trimethoxyphenyl)tetrahydrofuran, shown structurally as follows:

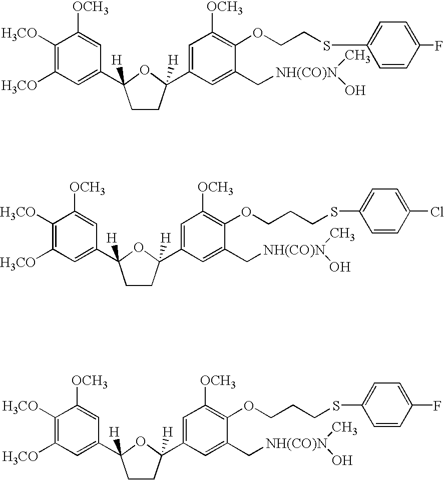

The active agent may be in the form of a pharmaceutically acceptable salt, ester, amide, prodrug, or other derivative or analog, or it may be modified by appending one or more appropriate functionalities to enhance selected biological properties. Such modifications are known in the art and include those which increase the rate of penetration into the skin or mucosal tissue, increase bioavailability, increase solubility, and the like. The active agent may be in enantiomerically pure or enantiomerically enriched form, or it may be incorporated into the formulations herein as a racemic mixture of enantiomers.

The active agent may be converted into a pharmaceutically acceptable salt using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992).

Acid addition salts are prepared from the free base using conventional means, involving reaction with a suitable acid. Typically, the base form of the compound is dissolved in a polar organic solvent such as methanol or ethanol and the acid is added at a temperature of about 0° C. to about 100° C., preferably at ambient temperature. The resulting salt either precipitates or may be brought out of solution by addition of a less polar solvent. Suitable acids for preparing acid addition salts include both organic acids, e.g., acetic acid, propionic: acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base.

Basic salts of acid moieties which may be present on the active agent (e.g., carboxylic acid groups) cam be prepared in a similar manner using pharmaceutically acceptable inorganic or organic bases. Examples of inorganic bases include ammonia and carbonates, hydroxides and hydrogen carbonates of group I and group II metals such as sodium, potassium, magnesium and calcium. Examples of organic bases include aliphatic and aromatic amines such as methylamine, trimethylamine, triethylamine, benzylamine, dibenzylamine or α- or β-phenylethylamine, and heterocyclic bases such as piperidine, 1-methylpiperidine and morpholine.

The active agent may also be converted into a pharmaceutically acceptable ester. Suitable esters include branched or unbranched, saturated or unsaturated C₁ to C₆ alkyl esters, for example, methyl, ethyl and vinyl esters.

Preparation of esters involves functionalization of hydroxyl and/or carboxyl groups which may be present within the molecular structure. Esters are typically acyl-substituted derivatives of free alcohol groups, i.e., moieties which are derived from carboxylic acids of the formula RCOOH where R is alkyl, and preferably is lower alkyl. Pharmacologically acceptable esters may be prepared using methods known to those skilled in the art and/or described in the pertinent literature. Esters can be reconverted to the free acids, if desired, by using conventional hydrogenolysis or hydrolysis procedures. Preparation of amides and prodrugs can be carried out in an analogous manner.

Other derivatives and analogs of the active agents may be prepared using standard techniques known to those skilled in the art of synthetic organic chemistry, or may be deduced by reference to the pertinent literature.

The 2,5-diaryl tetrahydrofurans, tetrahydrothiophenes, and pyrrolidines, 1,3-cyclopentanes, and the 2,4-diaryl tetrahydrofurans, tetrahydrothiophenes and pyrrolidines which serve as the active agents herein exhibit a number of stereochemical configurations. Carbon atoms 2 and 5 (or 2 and 4, in the compounds of Formula II) in the center ring are chiral, and thus the center ring exists at a minimum as a diastereomeric pair. Each diastereomer exists as a set of enantiomers. Therefore, based on the chiral C₂ and C₅ (or C₂ and C₄, in Formula II) atoms alone, the compound is a mixture of four enantiomers.

If nonhydrogen substituents are located on carbon atoms 3 and 4 in the center ring (or carbon atoms 3 and 5, in Formula II compounds) then the C₃ and C₄ atoms are also chiral, and can also exist as a diastereomeric pair, that is, also as a mixture of four enantiomers.

The R groups in the active compounds described herein can likewise include chiral carbon atoms and thus, optically active centers. It is sometimes found that one or more enantiomers of a biologically active compound is more active, and perhaps less toxic, than other enantiomers of the same compound. Such enantiomerically enriched compounds are often preferred for pharmaceutical administration to humans. For example, it has been discovered that trans-2,5-diaryl tetrahydrothiophene and trans-2,5-diaryl tetrahydrofuran are often more active PAF receptor antagonists than their cis counterparts. Synthesis of the pure trans isomer of CMI-392 is described in the examples herein.

Classical methods of resolving isomers include a variety of physical and chemical techniques. Often the simplest and most efficient technique is repeated recrystallization. Recrystallization can be performed at any stage in the preparation of the compound or the final enantiomeric product. If successful, this simple approach represents a method of choice. When recrystallization fails to provide material of acceptable optical purity, other methods can be evaluated. If the compound is basic, one can use chiral acids that form diastereomeric derivatives that may possess significantly different solubility properties. Nonlimiting examples of chiral acids include malic acid, mandelic acid, dibenzoyl tartaric acid, 3-bromocamphor-8-sulfonic acid, 10-camphorsulfonic acid and di-p-toluoyltartaric acid. Similarly, acylation of a free hydroxyl group with a chiral acid also results in the formation of diastereomeric derivatives whose physical properties may differ sufficiently to permit separation.

A first method for synthesizing CMI-392 is described in detail in Example 1. Briefly, the method involves synthesis of 3,4,5-trimethoxyphenylvinylketone from 3,4,5-trimethoxy-acetophenone, reaction of the vinyl ketone with an appropriately substituted iodobenzaldehyde, reduction of the dione so produced with sodium borohydride, and successive synthesis of the two side chains, (1) the para-chlorophenylthioethoxy group, followed by (2) the N′-methyl-N′-hydroxy-ureidylmethyl moiety. The method readily lends itself to scale-up, as demonstrated in Example 4.

A preferred form of the active agent is a novel crystalline form now provided using the synthetic methods, purification techniques and reagents described infra. For CMI-392, the novel crystalline form of the drug may be characterized as follows: appearance—white crystals; sharp melting point at 59.8° C.; TLC 60% ethyl acetate/n-hexane, R_{f}=0.35; stable in moist air; soluble and stable in aqueous solutions and water-containing formulations. One method for synthesizing and purifying this novel crystalline form of the active agent herein is set forth in Example 2. Alternative methods of preparing pure crystalline CMI-392 using acetovanillone or aspirin as starting materials are described in the Examples and Drawings which follow. It will be appreciated by those skilled in the art of synthetic organic chemistry that CMI-392 analogs and derivatives, particularly those compounds having Formula (I), (II) or (III), may also be synthesized using the methods described in the present examples, by making minor modifications to the starting compound(s) and/or reactants used.

With respect to preparation of the crystalline form of the active agent, and purification thereof, it is important to note that the choice of recrystallization solvent is key. The inventors herein have now found that isopropyl alcohol, optionally combined with up to 10% (v/v) n-hexane, provides for optimum recrystallization, giving rise to the aforementioned crystalline product in a form that is at least approximately 97% pure. Recrystallization is preferably followed by decolorization with activated carbon. While the active agents disclosed herein were not, as previously prepared, stable in air or moisture, or stable in formulations containing water or hydrophilic protic solvents (e.g., alcohols or the like), the novel crystalline form of the drug may be incorporated into pharmaceutical compositions containing water, alcohols or the like. Thus, creams, gels, solutions and the like may now be prepared using the presently disclosed active agents in crystalline form. With the prior form of the drug, only formulations which were essentially anhydrous (e.g., ointments) could be used. Thus, the novel form of the drug provides a significant advantage in utility.

Formulations:

A. Pharmaceutical Ointments

In one embodiment of the invention, a pharmaceutical formulation is provided as an ointment containing an active agent having the structural formula (I), (II) or (III) as defined in the preceding section. The ointment contains approximately 0.01 to 10 wt. %, preferably 0.5 to 8 wt. %, more preferably 4 wt. % to 8 wt. %, and optimally 4 wt. % to 6 wt. %, active agent, which may or may not be in crystalline form. The ointment also contains a skin penetration enhancer or a combination of enhancers for increasing the rate at which the active agent permeates into and/or through the skin or mucosal tissue. Preferably, the enhancer also stabilizes the drug, i.e., renders it less sensitive to heat and/or moisture. When the active agent in the ointment composition in a form other than the novel crystalline structure described in the preceding section, the ointment will contain less than about 5 wt. %, preferably less than about 1 wt. %, most preferably less than about 0.5 wt. %, protic solvents that are liquids at temperatures of less than about 30° C., e.g., water, lower alkanols, and the like. When the active agent is not in crystalline form, then, it is necessary that the ointment be essentially anhydrous.

A preferred ointment contains an enhancer composition comprising at least one component which is a saturated monofunctional or polyfunctional ester which may be either open-chain or cyclic. Such compounds have been found to enhance the stability of the active agent herein. That is, such esters not only result in a formulation which is chemically and physically stable, but, surprisingly, provide a formulation having greater stability than exhibited by the active agent alone. In addition, saturated monofunctional or polyfunctional esters serve as delivery aids with the potential to increase the permeation of the active agent into and/or through the skin or mucosal tissue.

Suitable ester components for incorporation into the enhancer composition are nontoxic organic compounds that are physically and chemically compatible with the active agent and in which the active agent has at least some solubility. Preferred esters are liquid at room temperature and have a molecular weight of less than about 250. Typical esters contain 3-18 carbon atoms and one to three ester functionalities, and are generally lower alkyl esters or cyclic esters. Particularly preferred esters are diethyl succinate, propylene carbonate (PC), diisopropyl adipate (DIA) and triacetin (also known as glyceryl triacetate). In fact, it has been found that these latter four esters work exceptionally well in combinations, to provide optimal enhancement of stability. Generally, the pharmaceutical formulations herein contain on the order of 0.02 wt % to 50 wt. %, preferably on the order of 0.02 wt. % to 20 wt. %, of the enhancer composition. For a formulation containing approximately 6 wt. % active agent, the agent should be at least about 15% (w/w) soluble in the ester component.

In this embodiment, the active agent and enhancer composition are present in an ointment base. As known in the art, ointments are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment base, may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, lanolin and stearic acid. Preferred water-soluble ointment bases are prepared from polyethylene glycols of varying molecular weight; again, reference may be had to Remington: The Science and Practice of Pharmacy for further information. Any of the aforementioned ointment bases may be used herein, although white petrolatum is preferred.

In addition to the active agent, the enhancer composition, and the ointment base, the formulation may contain various additives, known to those skilled in the art. Examples of additives include emulsifiers, solubilizing agents, opacifiers, anti-oxidants, anti-microbial agents, gelling agents, thickening agents, stabilizers, and the like.

Preparation of ointments will employ conventional techniques of drug formulation, particularly topical drug formulation, which are within the skill of the art. Such techniques are fully explained in the literature. See Remington: The Science and Practice of Pharmacy, cited supra, as well as Goodman & Gilman's The Pharmacological Basis of Therapeutics, 9th Ed. (New York: McGraw-Hill, 1996). Generally, the ointment base, e.g., petrolatum or the like, is warmed, combined with all components to be incorporated into the final formulation, and mixed thoroughly. The active agent is typically, although not necessarily, dissolved in the ester penetration enhancer and added last. After sufficient homogeneity has been achieved, the ointment is cooled. It may be desirable to perform the process under an inert atmosphere, e.g., under argon.

B. Other Formulations

In another embodiment of the invention, pharmaceutical formulations are provided that may or may not be ointments, and that contain the crystalline form of the active agent. In crystalline form, as explained elsewhere herein, the active agent is less sensitive to moisture and chemical attack by protic solvents or reagents. Accordingly, in this embodiment, a variety of formulation types are provided containing any number of carriers, vehicles, enhancers, and the like.

Pharmaceutical formulations containing the active agent in crystalline form may be in the form of a lotion, cream, paste, gel, solution, oil, powder, suppository, ointment or the like. The formulations contain carriers, excipients and the like which are generally suited to topical drug administration, including any such materials known in the art. It is essential, clearly, that the selected carrier not adversely affect the active agent or other components of the formulation. Examples of suitable topical carriers for use in combination with the crystalline form of the active agent include, but are not limited to, water, alcohols and other nontoxic organic solvents, glycerin, mineral oil, silicone, petroleum jelly, lanolin, fatty acids, vegetable oils, and waxes.

Lotions are preparations to be applied to the skin or muscosal surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like.

Creams, as known in the art, are viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington, supra, is generally a nonionic, anionic, cationic or amphoteric surfactant.

As indicated above, oil formulations of the invention are preferred. In particular, oil formulations that contain an alcohol component, especially a C₁₋₁₂ alcohol, or more preferably a C₁₋₈ or C₁₋₆ alcohol are preferred.

Branched alkyl alcohols, such as alcohols that have one, two, three or more secondary and/or tertiary carbon and/or alcohol groups, also are preferred for use in oil compositions of the invention. Use of isopropyl alcohol in oil compositions of the invention can provide unexpected storage stability. See, for instance, the results of Example 15 which follows. Preferred oil formulations contain from 1 to about 30 weight percent of isopropyl alcohol or other branched alkyl alcohol based on total weight of the formulation, more preferably from about 5 to about 15 weight percent of isopropyl alcohol or other branched alkyl alcohol based on total weight of the formulation, still more preferably about 10 weight percent of isopropyl alcohol or other branched alkyl alcohol based on total weight of the formulation. Other suitable components of oil compositions of the invention include e.g., propylene glycol dicaprylate/dicaprate (available under tradename of Miglyol 840), ethyl oleate, triacetin, and diisopropyl adipate caprylic/capric triglyceride (available under tradename of Miglyol 812).

The formulation may also be in the form of a gel. As will be appreciated by those working in the field of topical drug formulation, gels are semisolid, suspension-type systems. Single-phase gels contain organic macromolecules distributed substantially uniformly throughout the carrier liquid, which is typically aqueous, but also, preferably, contain an alcohol and, optionally, an oil.

Solution formulations are typically sprayed on to the skin using conventional means, as will be appreciated by those skilled in the art.

The pharmaceutical compositions of the invention may also be in the form of suppositories for rectal administration. These can be prepared by mixing the agent with a suitable non-irritating excipient which is solid at room temperature but liquid at the rectal temperature and therefore will melt in the rectum to release the drug. Such materials include cocoa butter, beeswax and polyethylene glycols. Compositions for administration to the colon may also be in the form of enema solutions, comprising standard vehicles used for such solutions, typically aqueous vehicles.

In the preferred formulations of the invention, the active agent is present in an amount representing 0.01 to 10 wt. %, preferably 0.5 to 8 wt. %, more preferably 4 wt. % to 8 wt. %, and optimally 4 wt. % to 6 wt. %, of the composition. Also, it is desirable that an enhancer composition be incorporated as described in part (A) of this section, containing one or more esters. It is also preferred that the composition contain some isopropyl alcohol, generally up to about 10 wt. %, preferably in the range of 0.1 wt. % to 10 wt. %, more preferably in the range of 0.5 wt. % to 7.5 wt. %, as this has been found to enhance the stability of the formulation.

In this embodiment, wherein the active agent used to prepare the formulation is in crystalline form, other components may be incorporated into the formulations as well, i.e., vehicles and additives which could react with or otherwise destabilize the “original” form of the drug (i.e., the waxy solid discussed earlier herein). For example, standard permeation enhancers can be included, such as, for example, dimethylsulfoxide (“DMSO”), dimethyl formamide (“DMF”), N,N-dimethylacetamide (“DMA”), decylmethylsulfoxide (“C₁₀MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone from Durham Pharmaceuticals, Durham, N.C.), ethanol, and the like. Other types of additives can be included as well, as discussed in the preceding section, i.e., solubilizing agents, opacifiers, anti-oxidants, etc.

The present pharmaceutical formulations, in this embodiment of the invention, may also be delivered to the skin using conventional “transdermal” patches, wherein the formulation is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable adhesive material that serves to affix the system to the skin during drug delivery. Examples of suitable adhesive materials include, but are not limited to, polyethylenes, polysiloxanes, polyisobutylenes, polyacrylates, polyurethanes, and the like. The adhesive selected will depend on the particular drug, vehicle, etc., i.e., the adhesive must be compatible with all components of the drug-containing composition. In an alternative embodiment, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates serves as the upper surface of the device. The material selected for the backing material should be substantially impermeable to the drug and any other components of the drug-containing composition, thus preventing loss of any components through the upper surface of the device. The backing layer may be either occlusive or nonocclusive, depending on whether it is desired that the skin become hydrated during drug delivery. The backing is preferably made of flexible elastomeric material, e.g., polyethylene, polypropylene, polyesters, or the like. During storage and prior to use, the laminated structure includes a release liner. Immediately prior to use, this layer is removed from the device so that the system may be affixed to the skin. The release liner should be made from a drug/vehicle impermeable material. These topical formulations and patches may be prepared using conventional techniques known to those skilled in the art or described in the pertinent literature.

Treatment of Inflammatory and/or Immune Disorders:

The pharmaceutical formulations of the invention are useful for treating humans and animals suffering from inflammatory and/or immune disorders, and, in particular, disorders mediated by PAF or products of 5-lipoxygenase. For example, the compositions find utility in the treatment in inflammatory skin disorders, including, but not limited to, psoriasis, contact dermatitis, atopic dermatitis (also known as allergic eczema), exfoliative dermatitis, seborrheic dermatitis, erythemas (including erythema multiforme and erythema nodosum), discoid lupus erythematosus and dermatomyositis. The compounds and compositions of the invention are particularly effective in treating psoriasis and atopic dermatitis. The formulations are administered topically, to the skin, mucosal tissue or eye, as ointments, creams, gels, solutions, oils, or the like, as described in the preceding section, within the context of a dosing regimen effective to bring about the desired result. The formulations may also be administered to the colon, typically as an ointment, enema solution, or suppository, to treat inflammation of the gastrointestinal tract. The preferred dose of active agent is in the range of about 0.01 to 300 mg/kg/day, preferably 0.1 to 100 mg/kg/day, more typically about 0.5 to 25 mg/kg/day. It will be recognized by those skilled in the art that the optimal quantity and spacing of individual dosages will be determined by the nature and extent of the condition being treated, the site of administration, and the particular individual undergoing treatment, and that such optimums can be determined by conventional techniques. It will also be appreciated by one skilled in the art that the optimal dosing regimen, i.e., the number and frequency of doses, can be ascertained using conventional course of treatment determination tests. Generally, a dosing regimen involves administration of the selected topical formulation at least once daily, and preferably one to four times daily, until symptoms have subsided.

It is to be understood that while the invention has been described in conjunction with the preferred specific embodiments thereof, the foregoing description, as well as the examples which follow, are intended to illustrate and not limit the scope of the invention. Other aspects, advantages and modifications will be apparent to those skilled in the art to which the invention pertains.

The following examples are put forth so as to provide those of ordinary skill in the art with a complete disclosure and description of how to make and use the compounds of the invention, and are not intended to limit the-scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric. All solvents were purchased as HPLC grade and, where appropriate, solvents and reagents were analyzed for purity using common techniques. All reactions were routinely conducted under an inert atmosphere of argon, unless otherwise indicated.

All patents, patent applications and publications cited herein are incorporated by reference in their entireties.

### EXAMPLE 1

### Synthesis of CMI-392

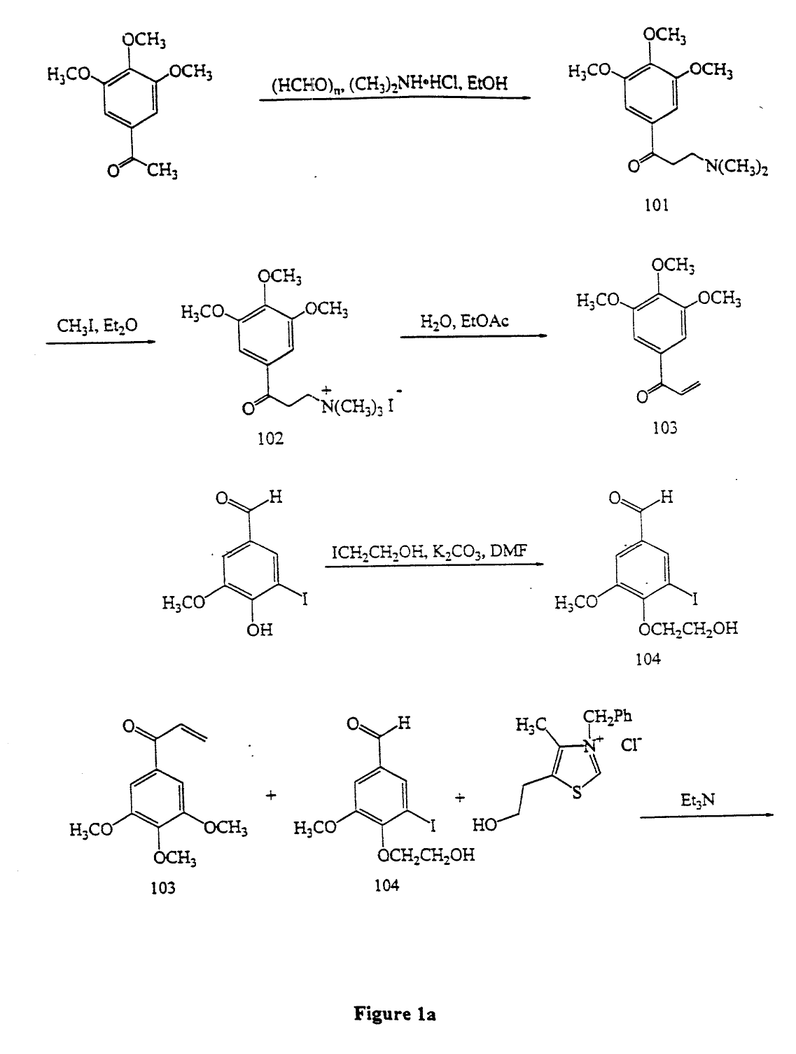

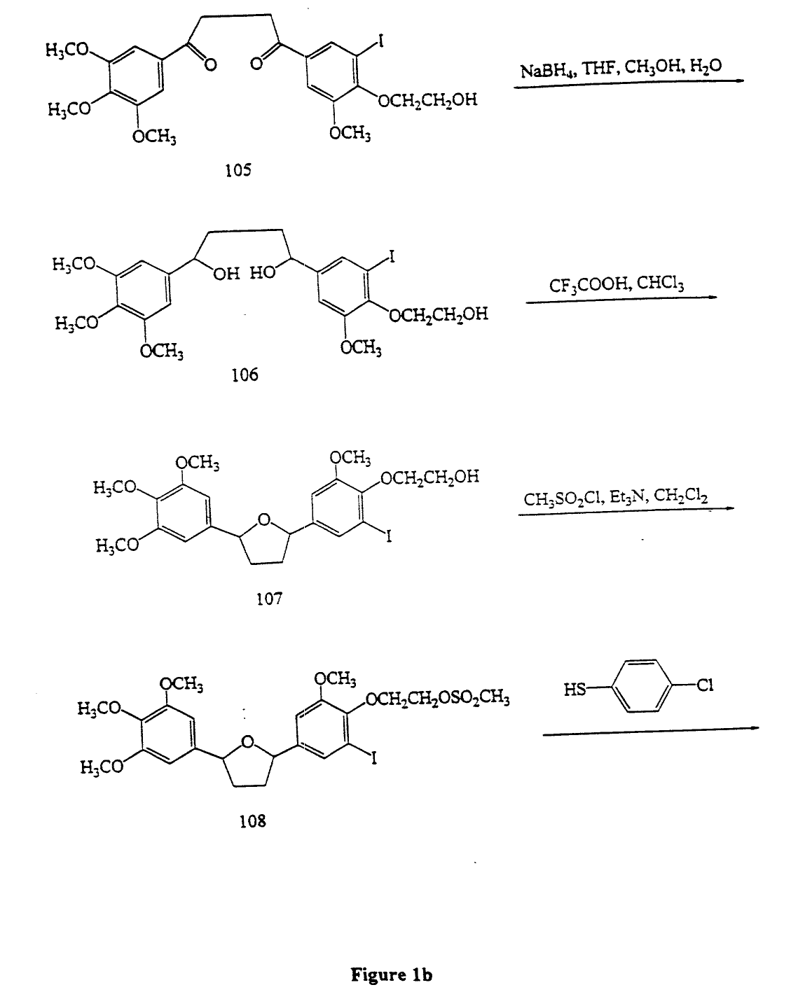

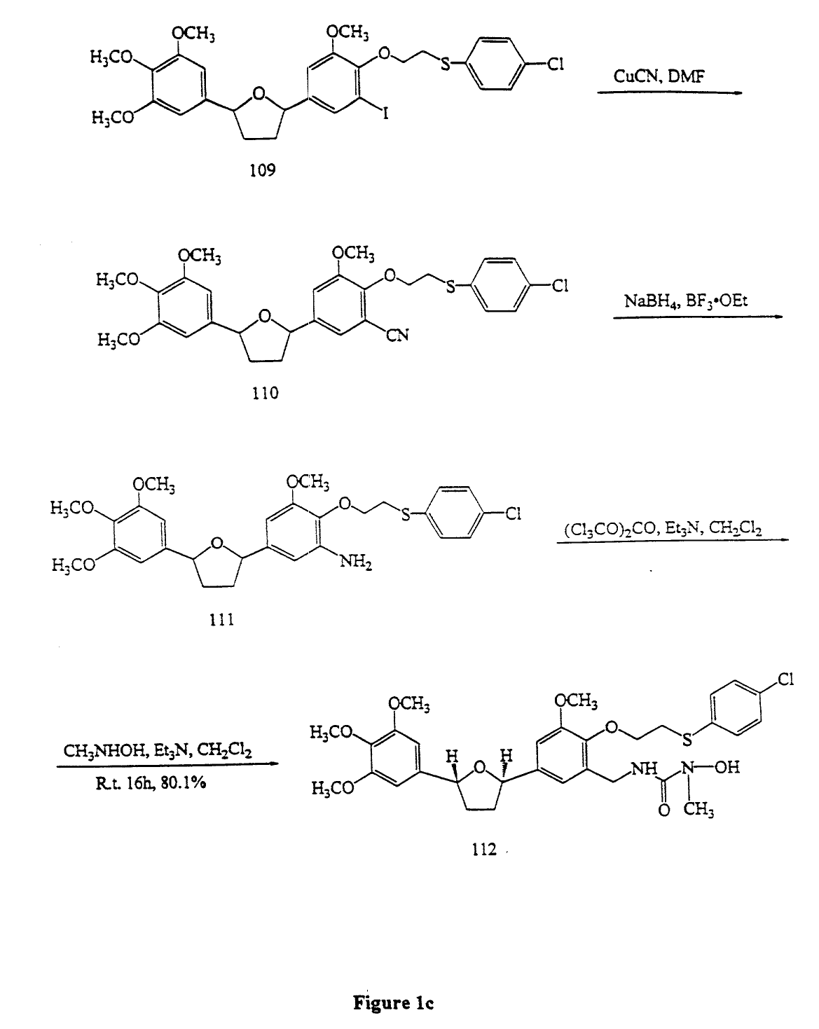

CMI-392, (±) trans-2-[5-(N-methyl-N⁰-hydroxyureidylmethyl)-3-methoxy-4-p-chlorophenylthioethoxyphenyl]-5-(3,4,5-trimethoxyphenyl)-tetrahydrofuran, was prepared using the synthesis illustrated schematically in FIGS. 1a through 1c, as follows:

(a) 3-(N,N-Dimethyl amino)-1-(3,4,5-trimethoxyphenyl)-1-propanone (compound 101): 3,4,5-Trimethoxyacetophenone (50 g, 237.8 mmole), paraformaldehyde (9.75 g, 304.7 mmole), dimethylamine hydrochloride (26.42 g, 324.0 mmole) and 5 mL conc. HCl were dissolved in 200 mL absolute ethanol and refluxed for 10 hours. Additional dimethylamine hydrochloride (13.21 g, 162.0 mmole) and paraformaldehyde (9.75 g, 304.7 mmole) were added and the solution returned to reflux. After 54 hours (total reaction time), 80 mL of 10% HCl and 500 mL of water were added and the solution was extracted with ethyl ether. The acidic aqueous layer was adjusted to pH 10 with 10% NaH. The basic solution was extracted with ethyl acetate, dried over MgSO4, filtered and evaporated in vacuo to provide 57.5 g of a yellow oil (92%). ¹H NMR (CDCl₃): 2.30 (s, 6H); 2,74 (t, 2H); 3.11 (t, 3H); 3.9 (s, 9H); 7.23 (s, 1H); 7.32 (s, 1H).

(b) 3-(N,N,N-Trimethylamino)-1-(3,4,5-trimethoxyphenyl)-1-propanone iodide (compound 102): 3-(N,N-Dimethylamino)-1-(3,4,5-trimethoxyphenyl)-1-propanone (57 g, 213.5 mmole) was dissolved in 200 mL of anhydrous diethyl ether. To this solution was added methyl iodide (57.6 g, 405.7 mmole). A white precipitate formed immediately, and the reaction mixture was stirred at room temperature for an additional 2 hours. This product was isolated by suction filtration (83.8 g, 96%).

(c) 3,4,5-Trimethoxyphenylvinylketone (compound 103): 3-(N,N,N-trimethylamino)-1-(3,4,5-trimethoxyphenyl)-1-propanone iodide (50 g, 120 mmole) was dissolved in H₂O (500 mL) and ethyl acetate (500 mL) was added. The mixture was vigorously stirred at reflux for 3 hours. The reaction mixture was cooled and the layers were separated. To the aqueous phase was added ethyl acetate (400 mL). This was brought to reflux for 1.5 hours. The reaction mixture was cooled and separated. The combined organic layers were washed with saturated NaCl) solution, dried over Na₂SO₄, filtered and concentrated in vacuo to an oil which was purified by flash column chromatography using 3:1 hexane/ethyl acetate as eluant (14.7 g, 54%). ¹H NMR (CDCl₃): 3.92 (s, 9H); 5.92 (d, 1H); 6.44 (d, 1H); 7.12 (m, 1H); 7.22 (s, 2H).

(d) 3-Methoxy-4-hydoxyethoxy-5-iodobenzaldehyde (compound 104): 5-Iodovanillin (25 g, 90 mmol) in DMF (100 mL) was added to potassium carbonate (18.6 g, 135 mmol). The mixture was heated at 40° C. for 16 hours. The reaction mixture was allowed to cool to room temperature, quenched with water (500 mL), and extracted with ethyl acetate. The organic layer was washed with water and saturated NaCl solution, dried over MgSO₄, filtered and evaporated in vacuo to an oil, and then purified by column chromatography (silica, 2:1 hexane/ethyl acetate), to provide the product (16.6 g, 57%). ¹H NMR (CDCl₃): 2.70 (t, 1H); 3.92 (t, 2H); 3.92 (s, 3H); 3.94 (s, 3H); 4,29 (t, 2H); 7.44 (s, 1H); 7.87 (s, 1H); 9.85 (s, 1H).

(e) 1-(3-Methoxy-4-hydroxyethoxy-5-iodophenyl)-4-(3,4,5-trimethoxyphenyl)-1,4-butanedione (compound 105): 3,4.5-Trimethoxyphenylvinylketone (4.8 g, 21.6 mmol), 3-methoxy-4-hydroxyethoxy-5-iodobenzaldehyde (5.7 g, 17.8 mmol), and 3-benzyl-5-(2-hydroxyethyl)-4-methylthiazolium chloride (1.9 g, 7.0 mmol) were stirred in triethylamine (20 mL) at 60° C. for 16 hours. The reaction mixture was then acidified with 10% HCl and extracted with dichloromethane. The organic layer was dried over MgSO₄, filtered and evaporated in vacuo. The product was purified in column chromatography (silica, 1:1 hexane/ethyl acetate) as a solid (9.7 g, 5 1%). ¹H NMR (CDCl₃): 3.41 (m. 4H); 3.90 (m, 2H); 3.92 (s, 3H); 3.93 (s, 9H); 4.26 (t, 2H); 7.29 (s, 2H); 7.57 (d, 1H); 8.08 (d, 1H).

(f) 1-(3-Methoxy-4-hydroxyethoxy-5-iodophenyl)-4-(3,4,5-trimethoxyphenyl)-1,4-butanediol (compound 106): 1-(3-Methoxy-4-hydroxy-ethoxy-5-iodophenyl)-4-(3, 4,5-trimethoxyphenyl)-1,4-butanedione (11.6 g, 21.3 mmol), was added to 120 mL tetrahydrofuran and 240 mL methanol. To this solution was added dropwise sodium borohydride (1.45 g, 38.4 mmol). in 60 mL water. The reaction mixture was stirred at room temperature for 2.5 hours, and then cooled, quenched with water, and the aqueous layer extracted with ethyl acetate. The organic layer was dried over MgSO₄, filtered and evaporated in vacuo to provide the product (11.8 g, 98.8%). ¹H NMR (CDCl₃): 1.84 (m, 4H); 3.84 (m, 2H); 3.86 (s. 3H); 3.87 (s, 9H); 4.15 (t, 2H); 4.68 (m, 2H); 6.57 (s, 2H); 6.91 (s, 1H); 7.32 (s, 1H).

(g) Trans-2-(3-Methoxy-4-hydroxyethoxy-5-iodophenyl)-5-(3,4,5-trimethoxyphenyl)-tetrahydrofuran (compound 107): To 1-(3-methoxy-4-hydroxyethoxy-5-iodophenyl)-4-(3,4,5-trimethoxyphenyl)-1,4-butanediol (11.8 g, 21.5 mmol) in chloroform (100 mL) at 0° C. was added dropwise trifluoroacetic acid (9.82 g, 86.1 mmol) in chloroform (100 mL) over 30 minutes. The solution was stirred at 0° C. for 2 hours and then at room temperature for 1 hour. The reaction mixture was quenched with 1N NaH and chloroform (100 mL) was added. The organic layer was washed with 1N NaH solution, water and saturated NaCl solution, and then dried over MgSO₄, filtered and evaporated in vacuo to an oil which was a cis and trans mixture.

The trans isomer was isolated by column chromatography (silica, 1:1 hexane/ethyl acetate) (4.7 g, 41.4%) as the faster eluting isomer. ¹H NMR (CDCl₃): 1.99 (m, 2H); 2.47 (m, 2H); 3.83 (t, 2H); 3.84 (s, 3H); 3.87 (s, 3H); 3.89 (s, 6H); 4.16 (t, 2H); 5.18 (m, 2H); 6.62 (s, 2H); 6.96 (d, 1H); 7.39 (d, 1H).

(h) Trans-2-(3-Methoxy-4-methylsulfoxyethoxy-5-iodophenyl)-5-(3,4,5-trimethoxyphenyl)tetrahydrofuran (compound 108): To the solution of trans-2-(3-methoxy-4-hydroxyethoxy-5-iodophenyl)-5-(3,4,5-trimethoxyphenyl) tetrahydrofuran (4.7 g, 8.87 mmol) in dichloromethane (50 mL) at 0° C. was added methylsulfonyl chloride (3.05 g, 26.6 mmole) and triethylamine (2.69 g, 26.60 mmol). The reaction mixture was stirred 0° C. for 2 hours and at room temperature overnight. The solvent was evaporated in vacuo and the residue purified by column chromatography (silica, 1:1 hexane/ethyl acetate) (4.17 g, 77.3%). ¹H NMR (CDCl₃): 1.98 (m, 2H); 2.45 (m, 2H); 3.15 (s, 3H); 3.84 (s, 3H); 3.88 (s, 9H); 4.26 (t, 2H); 4.61 (t, 2H); 5.17 (m, 2H); 6.62 (s, 2H): 6.96 (d, 1H); 7.38 (d, 1H).

(i) Trans-2-(3-Methoxy-4-p-chlorophenylthioethoxy-5-iodophenyl)-5-(3,4,5-trimethoxyphenyl)tetrahydrofuran (compound 109): Trans-2-(3-Methoxy-4-methylsulfoxyethoxy-5-iodophenyl)-5-(3,4,5-trimethoxyphenyl)tetrahydrofuran (2.5 g, 4.11 mmol) was dissolved in 50 mL ethanol. To this solution was added 4-chlorothiophenol (1.19 g, 8.22 mmol) and triethylamine (0.831 g, 8.22 mmol). The reaction mixture was refluxed for 16 hours and then the solvent was removed in vacuo. The residue was purified by column chromatography (silica, 3:1 hexane/ethyl acetate) (2.35 g, 87%). ¹H NMR (CDCl₃): 1.97 (m, 2H); 2.45 (m, 2H); 3.35 (t, 2H); 3.82 (s, 3H); 3.84 (s, 3H); 3.88.(s, 6H); 4.11 (t, 2H); 5.17 (m, 2H); 6.61 (s, 2H); 6.92 (s, 1H); 7.26 (d, 2H); 7.33 (d, 2H); 7.35 (s, 1H).

(j) Trans-2-(3-Methoxy-4-p-chlorophenylthioethoxy-5-cyanophenyl)-5-(3,4,5-trimethoxyphenyl)tetrahydrofuran (compound 110): Trans-2-(3-Methoxy-4-p-chlorophenyl-thioethoxy-5-iodophenyl)-5-(3,4,5-trimethoxyphenyl)tetrahydrofuran (2.35 g, 3.58 mmole) and CuCN (0.358 g, 4.30 mmole) in DMF (20 mL) were heated at 140 C for 16 hours. The reaction mixture was cooled and quenched with water and extracted with ethyl acetate. The organic layer was washed with water and saturated NaCl solution, dried over MgSO₄, filtered and evaporated in vacuo to an oil which was purified by column chromatography (silica, 2:1 hexane/ethyl acetate) (1.79 g, 90.0%). ¹H NMR (CDCl₃): 1.99 (m, 2H); 2.47 (m, 2H); 3.32 (t, 2H); 3.85 (s, 6H), 3.89 (s, 6H); 4.27 (t, 2H); 5.17 (m, 2H); 6.61 (s, 2H); 7.16 (s, 2H); 7.28 (d, 2H)—, 7.32 (d, 2H).

(k) Trans-Methoxy-4-p-chlorophenylthioethoxy-5-aminomethylphenyl)-5-(3,4,5-trimethoxyphenyl)tetrahydrofuran (compound 111): To trans-2-(3-methoxy-4-p-chlorophenyl-thioethoxy-5-cyanophenyl-5-(3,4,5-trimethoxyphenyl)tetrahydrofuran (300 mg, 0.5405 mmol) in tetrahydrofuran (THF) (10 mL) was added sodium borohydride (36.8 mg, 0.9729 mmol) and boron trifluoride etherate (191.8 mg, 1.3512 mmol) dropwise. The reaction mixture was refluxed for 1 hour, cooled, and then treated with a few drops of 10% HCl. The reaction mixture was poured into 10% K₂CO₃ and extracted with ethyl acetate. The organic layer was washed with water and saturated NaCl solution, dried over MgSO₄, filtered, and evaporated in vacuo to an oil which was purified by column chromatography (silica, 93:7 CH₂Cl₂/MeOH) (64 mg, 21.2%). ¹H NMR (CDCl₃): 1.99 (m, 2H); 2.46 (m, 2H); 3.28 (t, 21H); 3.84 (s, 6H); 3.88 (s, 6H); 4.26 (t, 2H): 5.19 (m, 2H); 6.71 (s, 2H), 6.90 (s, 2H); 7.25 (d, 2H); 7.32 (d, 2H).

(l) Trans-2-[5-(N′-Methyl-N′-hydroxyureidylmethyl)-3-methoxy-4-p-chlorophenyl-thioethoxyplhenyl]-5-(3,4,5-trimethoxyphenyl)tetrahydrofuran (112): Trans-2-(3-methoxy-4-p-chlorophenylthioethoxy-5-aminomethyl-phenyl)-5-(3,4,5-trimethoxyphenyl)tetrahydrofuran (54 mg, 0.0966 mmol) was dissolved in 4 mL dry dichloromethane. To this solution was added triphosgene (9.46 mg, 0.0319 mmol) and triethylamine (9.77 mg, 0.0966 mmol). The reaction mixture was refluxed for 2 hours and then cooled to room temperature. To this solution was then added triethylamine (35.2 mg, 0.3478 mmol) and methylhydroxylamine hydrochloride (24.2 mg. 0.2898 mmol). The reaction mixture was stirred at room temperature overnight, quenched with water and extracted with dichloromethane. The organic layer was washed with water and saturated NaCl solution, dried over MgSO₄, filtered and evaporated in vacuo. The product was purified by column chromatography (silica, ethyl acetate) (49 mg, 80.1%). ¹H NMR (CDCl₃): 1.97 (m, 2H); 2.43 (m, 2H); 3.08 (s, 3H); 3.27 (t, 2H); 3.82 (s, 3H); 3.83 (s, 3H); 3.87 (s, 6H); 4.15 (t, 2H); 4.39 (d, 2H); 5.17 (m, 2H); 6.41 (t, 1H); 6.51 (s, 2H); 6.78 (broad s, 1H); 6.90 (s, 2H); 7.24 (d, 2H); 7.31 (d, 2H).

### EXAMPLE 2

### Synthesis of CMI-392 in Crystalline Form

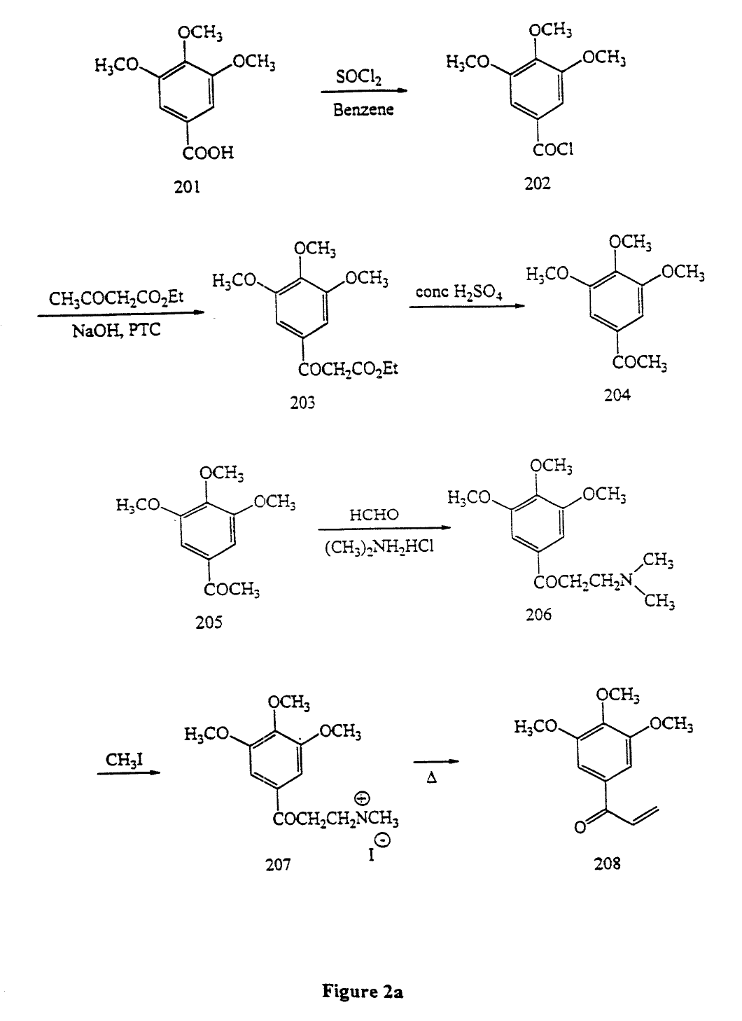

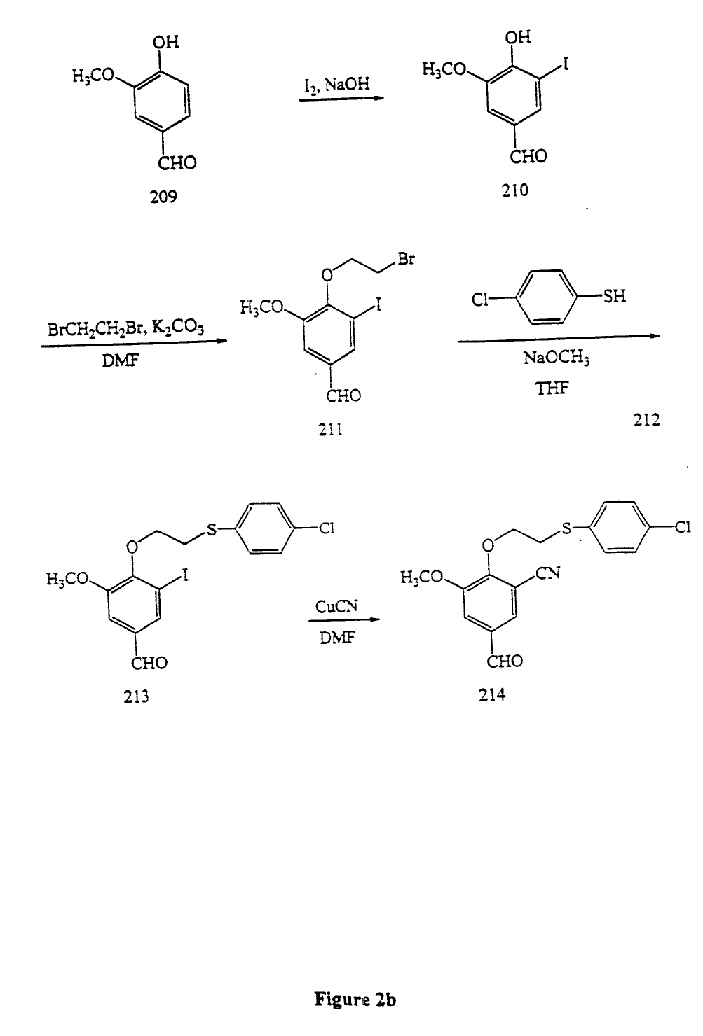

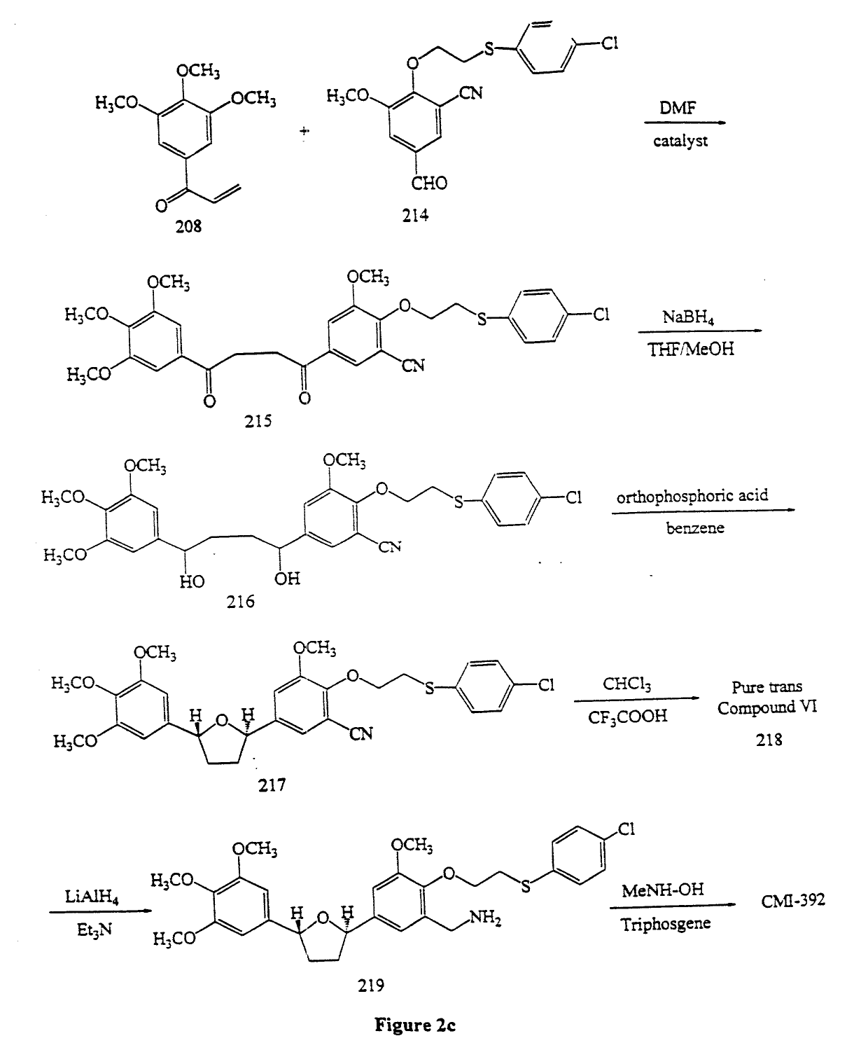

An alternative method for synthesizing CMI-392 was used as described in this example and as illustrated in FIGS. 2a through 2c. The final step of the procedure provides the compound in stable crystalline form. Quantities of all reagents were selected to provide 250 g product.

(a) 3,4,5-Trimethoxybenzoyl chloride (compound 202): In a 100 L, all glass assembly connected to an HCl absorbing tank, 3,4,5-trimethoxybenzoic acid (25.0 kg), SOCl₂ (12.5 L) and benzene (35.0 L) were added. To this solution, DMF (150 mL) was added and the contents stirred at room temperature until the acid went into solution (approximately 4 hours) and the reaction was almost complete. The solution was refluxed for 0.5 hours and the benzene along with the excess SOCl₂ was distilled off. The product 202 was crystallized from hexane. Yield: 24.0 kg. M.p.: 75° C.

(b) 3,4,5-Trimethoxybenzoyl acetate (compound 203): In a 250 L stainless steel reactor, toluene (17.6 L), ethyl acetoacetate (10 L), triethylbenzyl ammonium chloride (80 g) and water (60 L) were taken and contents cooled to 0-5° C. To the stirred solution sodium hydroxide (8 kg in 12 L water) was added in 30 minutes. The contents were stirred for an additional 1.5 hours. To this, trimethoxybenzoyl chloride (20 kg) dissolved in toluene (40 L) and the remaining aqueous NaH. (4 kg in 12 L) were simultaneously added in 5 hours. The contents were stirred for an additional 2 hours. The stirring was then stopped and the two layers separated. The aqueous layer was then heated with NH₄Cl (4.6 kg, and stirred for ten hours. The solid was then collected by centrifuge, washed and dried at room temperature. Yield, 203: 8 kg. M.p.: 91-93° C.

(c) 3,4,5-Trimethoxyacetophenone (compound 204): In a 250 L, glass reactor, keto ester 203 obtained in the preceding example (7.0 kg), along with concentrated sulfuric acid, was refluxed for eight hours. The acidic solution was then allowed to reach room temperature and the solid was collected by filtration. Yield, compound 204: 4.5 kg (87%). M.p.: 76° C.

(d) 3,4,5-Trimethoxyphenyl-(N,N-dimethylaminoethyl)ketone (compound 205): In a 50 L all glass assembly, compound 204, paraformaldehyde (1.14 kg), dimethylamine HCl (2.32 kg), concentrated HCl (100 mL) and isopropanol (12.0 L) were combined and refluxed for three hours. Half of the isopropanol was distilled off and the contents were cooled. The filtrate was concentrated to obtain a second crop and acidified with concentrated HCl. The white crystalline solid obtained, compound If was collected and washed with acetone. Quantity obtained: 4.9 kg (86%). M.p.: 172-175° C.

(e) Quaternary ammonium salt 207: In a 50 L all glass assembly, compound 206 (3.5 kg) was stirred with 6 L of 10% aqueous NaH. The free base thus liberated was extracted with ethyl acetate (2×7.5 L). The organic layer was collected and cooled to 0-5° C. To this cold solution, MeI (1.045 L) was added and stirred for two hours at 0-10° C. and then at room temperature for an additional four hours. The solid that separated was filtered and dried under reduced pressure. Quantity obtained, compound 207: 4.1 kg (87.8%). M.p.: 165-170° C.

(f) 3,4,5-Trimethoxyphenylvinylketone (compound 208): Compound 207 (4.1 kg) was dissolved in 41 L of water in a 100 L glass assembly. To this was added ethyl acetate (12 L) and the contents were refluxed for two hours. The organic layer was collected and the aqueous layer was once again mixed with ethyl acetate (10 L) and refluxed for 1 hour. After separating the organic layer, the aqueous layer was subjected to the same treatment (10 L). All the organic extracts were combined and the solvent was removed by distillation. The crude vinyl ketone was filtered through a silica gel bed (2.5 kg) and eluted with methylene chloride. Removal of solvent gave a low m.p. compound (1.5 kg) which was used as is in the next step.

(g) 5-Iodovanillin (compound 210): Vanillin (5 kg) was dissolved in a 3.2% aqueous sodium hydroxide solution (1.60 kg dissolved in 50 L water) in a 100 L, all glass assembly. To this, while stirring (90 rpm), iodine crystals (8.70 kg) were added and the contents heated to 85-90 C for 8 hr. At the end, the reaction was monitored by TLC (solvent system: benzene) and showed traces of vanillin. The contents were cooled to room temperature and the solution was neutralized with HCl (150 mL). The solid, iodovanillin, was collected and centrifuged, and washed with water, followed by sodium dithionite (1.0 kg dissolved in 10 L water). The dark brown compound was dried in an oven (heated by dry air at 80° C.) for 3 hr. The crude dark colored iodovanillin (8.50 kg) was taken in a 50 L glass assembly along with isopropanol (17 L) and heated to reflux for 30 min. Half of the material went into solution. The contents were then cooled and the yellow product was collected by centrifuge. The pure iodovanillin was dried (90° C. for 6 hr). Quantity obtained: 6.5 kg. M.p.: 180-181° C. HPLC: >98% pure.

(h) 3-Methoxy-4-bromoethoxy-5-iodovanillin (compound 211): In a 50 L all glass assembly, 5-iodovanillin (5.0 kg) was dissolved in 25.0 L of DMF. Anhydrous K₂CO₃ (2.5 kg) was added, followed by 1,2-dibromoethane (5.0 L; 2.5 kg). The mixture was heated to 80-85° C. for a period of 3 hr. The reaction was monitored by TLC (solvent system: 30% ethyl acetate in hexane). The contents were allowed to cool to room temperature, and the K₂CO₃ was collected by filtration. The filtrate was transferred to a 20 L flask and the DMF distilled off along with an excess of dibromoethane below 50° C. (2 mm)(recovery >90%). The slurry was dissolved in dichloromethane (2.5 times) and washed with brine (2×25 L) to remove excess DMF. The dichloromethane solution was then taken and solvent removed. The product solidifies on cooling at 0° C. The crude solid was then stirred with hexane (10 L) containing methanol (1 L) to yield a yellow crystalline product, 211. Quantity: 5.1 kg. M.p., 73-75° C.

(i) 3-Methoxy-t-(p-chlorophenylethylthio)-oxy-5-iodobenzaldehyde (compound 213): Sodium methoxide (0.738 g) was added to 10 L THF in a 50 L all glass assembly. The contents were stirred at room temperature, and then cooled to 5° C. A solution of p-chlorothiophenol dissolved in THF (1.975 kg in 5 L of THF) was added over a period of 2 hr with constant stirring, while the temperature was maintained below approximately 10° C. To this was added a solution of compound 211 dissolved in THF (5.0 kg in 10 L). After 10 hr at room temperature, TLC confirmed the absence of starting material IIc (solvent system, 10% benzene in n-hexane). The solution was then quenched with a salt solution of NH₄Cl (5.0 L) and stirred for 15 min.

The THF layer was collected and the solvent removed. To this, dichloromethane (10 L) was added and the organic layer was washed with water (5.0 L). The solvent was removed and the thick mass solidified on cooling to 0° C. This was stirred with a mixture of 10 L of hexane containing 500 mL of MeOH to give a yellow solid. Quantity obtained, compound 213: 4.5-5.0 kg. HPLC: >90% pure. M.p., 63-65° C.

(j) 3-Methoxy-4-(p-chlorophenylethylthio)-oxy-5-cyanobenzaldehyde (compound 214): In a 20 L, three-necked flask DMF (6.0 L), CuCN (596 g) and compound 213 were heated, while stirring, to 120° C. for 3 hr. At the end of the reaction, the iodo compound 213 was converted to the cyano derivative 214 as shown by TLC (solvent system: 30% ethyl acetate in hexane). The contents were cooled to room temperature, treated with EDTA and then mixed with benzene (12 L). The soluble portion of the benzene layer was collected from the insoluble matter and washed with brine solution (6.0 L×4). The solvent was then removed to obtain crude cyano compound 214 (1.2-1.4 kg) which was then stirred with 4.0 L of benzene-hexane mixture (1:9) for 2 hr at room temperature. The pale yellow product 214 was collected (purity >90%) (1.1 kg). The product was used in the next step without further purification.

(k) 1-(3′,4′,5′-Trimethoxyphenyl)-4-[4′-(p-chlorophenylthioethyl)-oxy-5′-cyano-3′-methoxy]phenyl-1,4-dioxobutane (compound 215): Compound 214 (1.0 kg), prepared as described in the preceding section, was dissolved in DMF (2.5 L) in a 20 L round bottom flask. Catalyst (155 g), compound 208 (715 g) followed by TEA (600 mL) were added and the contents stirred for 1.5 hr at 70-75° C. The reaction mixture was then cooled to 30-40° C. and HCl (1.5 L) in water (5.0 L) was added slowly with stirring in 1 hr. The solid that separated was filtered off and washed with water (4.0 L) and dried under reduced pressure. Crude product was then dissolved in ethyl acetate (5.0 L) while boiling and n-hexane (3.0 L) was added until turbidity persisted. The contents were then cooled to room temperature and kept standing for 2-3 hr. The solid was filtered, washed with 10% ethyl acetate in petroleum ether (1.0 L) and dried under vacuum. Yield, compound 215: 1.2 kg (75%). M.p., 100-102° C.

(l) 1-(3′,4′,5′-Trimethoxyphenyl)-4-[4′-(p-chlorophenylthioethyl)-oxy-5′-cyano-3′-methoxy]phenyl-1,4-butanediol (compound 216): Compound 215 was dissolved in THF (6.0 L) in a 10 L flask and methanol (400 mL) was added. The contents were cooled to 0° C. and NaBH₄ (80 g) was added in 10 g portions over a period of 1.5 hr, while maintaining the temperature in the range of 0 to 10° C. Stirring was continued for 1.5 hr at below 20° C. The reaction mixture was then quenched with water (500 mL) and stirred for 0.5 hr. THF and methanol were removed and the residue dissolved in chloroform (3.0 L) and washed with water (1.0 L). The aqueous layer was again extracted with CHCl₃ (1 L) and the combined organic layers were washed with water (1.0 L). The organic layer was dried over sodium sulfate (500 g) and concentrated to give an oily compound. The compound was used without further purification in the next step. Yield, compound 216: 1.0 kg.

(m) Trans-2-(3-Methoxy-4-p-chlorophenylthioethoxy-5-cyanophenyl)-5-(3,4,5-trimethoxyphenyl)tetrahydrofuran (compound 217; note—same compound as 110 in Example 1, FIG. 1c): Compound 216 (1.0 g), prepared in the preceding step, was dissolved in benzene (5.0 L). Orthophosphoric acid (400 ML) was added and the contents refluxed for 2 hr with continuous removal of water. The contents were cooled to room temperature and kept standing for 0.5 hr. The upper benzene layer was taken in a separating funnel and washed with water (1.0 L) followed by NaHCO₃ solution (2×1.0 L). The phosphoric acid layer was extracted with benzene (1.0 L), and the benzene layer was washed with water (500 mL), NaHCO₃ solution (2×250 mL) and finally with water (500 mL). The combined benzene extracts were concentrated to obtain 1.0 kg of crude product. The residue was filtered through a silica gel column and eluted with ethyl acetate/n-hexane (40:60). Fractions containing compound were collected and concentrated to give an oily compound containing a cis-trans mixture of compound 217. Yield: 800 g (cis:trans ratio, 40:60).

(n) Crystallization of the cis-trans mixture of compound 217 to give pure trans compound 218: The mixture of cis-trans compound 217 (1.0 kg) obtained in the preceding step was dissolved in ethyl acetate (2.2 L) and n-hexane was added slowly with continuous shaking until turbidity of the solution persisted (˜3.0 L). The solution was then seeded with pure trans compound and left standing at −10° C. for a period of 15 hr. A white solid separated out which was filtered and washed with 20% ethyl acetate/n-hexane and then dried under reduced pressure. Yield: 250-300 g (>97% pure). M.p.: 86° C. The filtrate was concentrated arid the product was left for isomerization.

(o) Isomerization of cis to trans (218): Trifluoroacetic acid (200 mL) was added to the concentrated mixture of cis-trans compound 217 (1.0 g; obtained in step (m)) in chloroform (2.0 L). The mixture was stirred for 7-8 hr at room temperature. Water (2.0 L) was added and the organic layer was separated, washed with 20% NaHCO₃ soln (2×1.0 L) and finally with water (1.0 L). The chloroform was distilled off and the residue crystallized from ethyl acetate:n-hexane (1.0:3.0 L) to obtain a white crystalline solid. Yield: 240 g (>97%, pure trans). This procedure was repeated to collect maximum trans isomer (218).

(p) Trans-Methoxy-4-p-chlorophenylthioethoxy-5-aminomethylphenyl)-5-(3,4,5-trimethoxyphenyl)tetrahydrofuran (compound 219; note—same compound as 111 in Example 1, FIG. 1c): THF (1.5 L) and triethylamine (136 g; 188 mL) were taken in a 10 L flask fitted with a dropping funnel and a calcium chloride guard tube, and the contents were cooled to 0° C. Lithium aluminum hydride (35 g) was added in portions, while the temperature of the reaction mixture was maintained just below approximately 10° C., with constant stirring. Stirring was continued for 1 hr at 10-20° C. Compound 218 (500 g in 1.5 L THF) was added slowly. Additional lithium aluminum hydride (17 g) was then added and stirring was continued for 1 hr at <10° C. to complete the reaction. The reaction was monitored using TLC (solvent system: 10% methanol in CHCl₃). The contents were then cooled to 0° C. and sodium sulfate solution (150 g in 300 mL water) was added slowly while stirring, over a 15 minute period, followed by addition of NaCl (100 g). The inorganic salts were filtered off, extracted with boiling ethyl acetate (2×500 mL) and again filtered. The combined filtrates were concentrated and the residue purified by column chromatography over silica gel, using first benzene and then 2-6% methanol in chloroform as eluents. Compound-containing fractions were combined and concentrated to give an oily liquid. Yield: 350-400 g (˜75%).

(q) CMI-392, (±)trans-2-[5-(N′-methyl-N′-hydroxyureidylmethyl)-3-methoxy-4-p-chlorophenylthioethoxyphenyl]-5-(3,4,5-trimethoxyphenyl)tetrahydrofuran: Compound 219 (375 g) was dissolved in dichloromethane and cooled to 0° C. Triphosgene was added to the reaction mixture, followed by triethylamine (94 mL). The mixture was stirred for 3 hr at room temperature under nitrogen. The contents were then cooled to 0° C. and triethylamine (188 mL) was added, followed by addition of N-methylhydroxylamine HCl (84 g in 3 lots), with stirring, over a 15-16 hr period at room temperature. Water (2.0 L) was then added and the organic layer was separated. The aqueous layer was extracted with dichloromethane (1 L). The combined organic layers were washed with brine (1 L) followed by water (1 L). The organic layer was separated, dried over Na₂SO₄ and concentrated. The residue, a thick liquid (0.4 kg), was purified by column chromatography over silica gel using ethyl acetate/n-hexane as an eluent. Compound-containing fractions were combined and concentrated to give a thick oily compound. Yield: 250 g.

(r) Purification of CMI-392:

The oily product obtained in the preceding step was dissolved in isopropyl alcohol (5 times) and left −0° C. for 10 hr. The gummy yellow solid that settled at the bottom was separated from the upper white crystalline compound by filtration (65 g, purity >98%). The filtrate was added to the yellow solid and dissolved by heating. The contents were left at 0° C. After 10 hr, a second crop of white crystalline compound was collected (60 g, purity >95%/0). The crops were mixed and recrystallized to give ˜100 g of 98% pure compound.

The mother liquors were pooled and chromatographed over silica gel (2.5 kg) and eluted with isopropyl alcohol in hexane (2-10%).

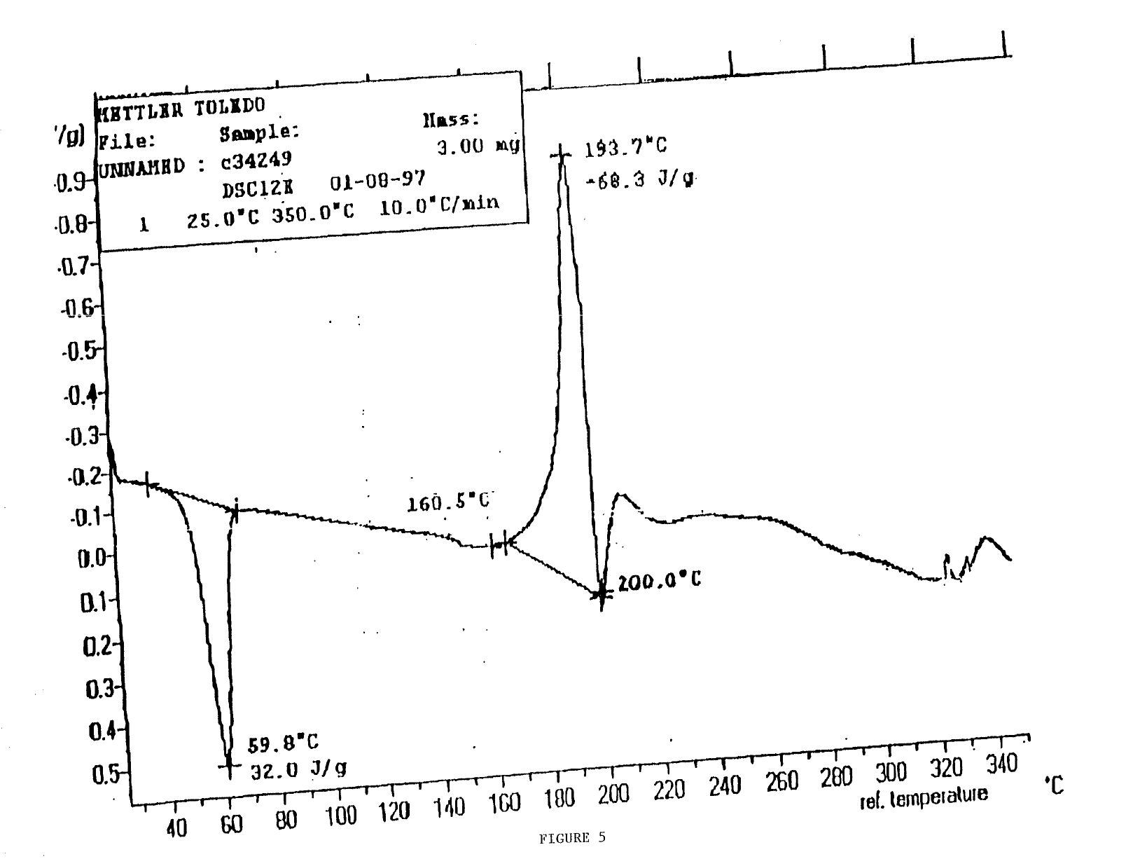

All fractions containing pure CMI-392 were collected. Removal of solvent and recrystallization gave the pure compound (50 g, purity >97%). The two crops were mixed from 4 batches, washed with n-hexane and dried under reduced pressure to give ˜512 g of CMI-392 containing 8% isopropyl alcohol. HPLC >98%. M.p., 59.8° C. A DSC of the crystalline compound (25.0° C. to 350.0° C., 10.0° C./min.) is shown in FIG. 5.

### EXAMPLE 3

### Alternative Synthetic Routes to Crystalline CMI-392

### (A) Using Acetovanillone as a Starting Material

(a) 5-Iodoacetovanillone (compound 302): Sodium hydrogen carbonate (657 g) was dissolved in water (8 L), acetovanillone (1.0 kg) was added and the solution stirred for 0.5 hours. Iodine (1.828 kg) was added in 10-15 g portions over a period of 2 hours, and the reaction mixture was stirred for 18-20 hours at room temperature. The reaction was monitored by TLC (silica gel, solvent system: benzene). The reaction solution was acidified with concentrated HCl (175 ml) bringing the pH to about 2, and the solution stirred for an additional hour. The solid was collected by filtration, washed with 20% sodium dithionite solution (5 L) and water (5 L), and dried for 12-14 hours at room temperature. The crude product was crystallized from isopropyl alcohol (2 L). Yield: 1.51 kg (85%), purity: 91% (HPLC), m.p.: 175-176° C.

(b) 4-[2-Bromoethoxy]-3-iodo-5-methoxyacetophenone (compound 303): To a 10 L three neck round bottom flash containing 5-iodovanillone (compound 302, 1.0 kg) dissolved in DMF (5 L) containing potassium carbonate (1.417 kg), was added 1,2-dibromoethane (2.57 kg). The solution was heated to 60-70° C. for 4-5 hours. The reaction was monitored by TLC (silica gel, solvent system: 30% ethyl acetate in n-hexane). The solution was cooled to room temperature and the solid collected by filtration and washed with benzene (500 mL). The filtrate was concentrated under reduced pressure. The residue was dissolved in benzene (3 L), washed with water (2×1 L) and saturated brine solution (2×1 L). The organic layer was dried over sodium sulfate (500 g) and concentrated under reduced pressure to give compound. Yield: 1.025 kg (75%), purity: 87% (HPLC), m.p.: 82-83° C.

(c) 3-Iodo-5-methoxy-4-[2-p-chlorothiophenylethoxy] acetophenone (compound 304): A 10 L three neck round bottom flask fitted with a calcium chloride guard tube and containing THF (2.5 L) was cooled to 0-5° C. and sodium methoxide (149 g) was slowly added over a 1 hour period. A solution of 0.362 kg p-chlorothiophenol in 1.0 L THF was then added over a 1-hr. period. The solution was stirred for another 1.5 hours at below 10° C., and compound 303 (1.0 kg) in THF (1.5 L) was then slowly added over a 1.5 hour period. The reaction was stirred at room temperature for 12-14 hours and monitored by TLC (silica gel, solvent system: 25% benzene in hexane). Saturated ammonium chloride (500 mL) was added, the solution was stirred for 1 hour, and the organic layer was separated and concentrated under reduced pressure. The residue was washed with water (2×2 L) and dried at room temperature for 24 hours. Yield: 1.08 kg (93%), purity: 90%, m.p.: 100-101° C.

(d) Mannich salt of 3-iodo-5-methoxy-4 [2-p-chlorothiophenylethoxy]acetophenone (compound 305): In a 5 L flask filter with a calcium chloride guard tube, compound 304 (500 g), paraformaldehyde (32 g), dimethylamine HCl (76 g) and concentrated HCl (20 mL) were combined and the contents refluxed for 2 hours. The reaction was monitored by TLC (silica gel, solvent system: 25% benzene in n-hexane). Paraformaldehyde (32 g) and dimethylamine HCl (76 g) were added to the reaction mixture twice, followed by reflux for 2 hours after each addition. The reaction was allowed to cool to room temperature, acetone (1.5 L) was added, and the reaction cooled to 0° C. for 4-5 hours. The solid was collected by filtration, washed with acetone (500 mL), and dried at room temperature for 2-3 hours. Yield: 325 g (54%), m.p.: 142-144° C.

(e) Quaternary ammonium salt of 3-iodo-5-methoxy-4-[2-p-chlorothiophenylethoxy] acetophenone (compound 306): Compound 305 (304 g) was dissolved in ethyl acetate (1.0 L) and then 3.5% solution of NaOH (1 L) was added. The reaction mixture was stirred for 0.5 hours, the organic layer was separated, and the aqueous layer extracted with ethyl acetate (2×250 mL). The organic layers were combined, washed with water (2×500 mL) and dried over sodium sulfate. The inorganic salts were separated by filtration. The organic filtrate was cooled to 0° C. in a 3 L round bottom flask and then methyl iodide (106 g) was added in three portions over 0.5 hours. The reaction mixture was then stirred at room temperature for 5-6 hours. The solid was collected by filtration and washed with ethyl acetate (500 mL). Yield: 310 g (81%), m.p.: 135-137° C.

(f) 3-Iodo-5-methoxy-4-(2-p-chlorothiophenylethoxy)phenyl vinyl ketone (compound 307): In a 5 L round bottom flask, compound (306) (300 g) was added to water (1.5 L) that was warmed to 35-40° C. Then, ethyl acetate (1.0 L) was added and the reaction solution refluxed for 1 hour. Upon cooling to room temperature, the organic layer was separated, and the aqueous layer was again refluxed with ethyl acetate (2×250 ML). The combined organic layers were dried over sodium sulfate and concentrated under reduced pressure. Yield: 186 g (86%), purity: 95% (HPLC), m.p.: 91-92° C.

(g) 1-(3′,4′,5′-Trimethoxyphenyl)-4-[3″-iodo-5″-methoxy-4″-(2-p-chlorothiophenyl-ethoxy)phenyl]-1,4-dioxobutane (compound 309): 3-Benzyl-5-(2-hydroxyethyl)-4-methylthiazolium chloride catalyst (45.5 g) and 3,4,5-trimethoxybenzaldehyde (compound 308, 165 g) were dissolved with stirring in DMF (1 L) in a 5 L round bottom flask containing a calcium chloride guard, and then compound 307 (400 g) was added. After about 0.5 hours of stirring, triethylamine (128 g) was slowly added and the reaction mixture heated to 70-80° C. until completion as determined by TLC (silica gel, solvent system: 40% ethyl acetate in n-hexane). The reaction mixture was then cooled to room temperature and 10% HCl (4 L) was added slowly with vigorous stirring for about 1 hour. The aqueous layer was decanted, and the product washed with water (2×2 L) with decantation. The crude product was stirred in isopropyl alcohol (1 L) for 1 hour, the solid collected by filtration and washed with isopropyl alcohol (500 1L). Yield: 425 g (75.2%), m.p.: 105-107° C.

(h) 1-[3′-Iodo-5′-methoxy-4′-(2-p-chlorothiophenylethoxy)phenyl-4-(3″,4″,5″-trimethoxyphenyl)-butan-1,4-diol (compound 310): Compound 309 (400 g) was dissolved in THF (2 L) and methanol (100 mL), and the 5 L round bottom flask was cooled to 0° C. NaBH₄ (25 g) was then added in 2-3 g portions over a period of 1 hour. Stirring was continued for 2 hours at below 10° C. The reaction was then quenched with a saturated solution of ammonium chloride (100 mL) and stirred for another hour. The solvents were removed under reduced pressure, benzene (1.5 L) and water (1.0 L) were added to the residue, the organic layer was separated, and the aqueous layer was extracted once again with benzene (0.5 L). The combined organic layers were washed with water (0.5 L) and then with brine (2×0.5 L), dried over sodium sulfate and filtered. The compound in the filtrate was used in the next step without further purification.

(i) Cis/trans-2-(:3′,4′,5′-Trimethoxyphenyl)-5-[3″-iodo-5″-methoxy-4-(2-p-chlorothiophenylethoxy)phenyl] tetrahydrofuran (compound 311). The benzene solution containing compound 310 (1,2 L), prepared in the preceding step, and orthophosphoric acid (130 mL) were placed in a 3 L round bottom flask and refluxed for 2 hours. The contents were cooled to room temperature and the upper benzene layer was decanted. The benzene layer was washed with water (500 mL), 20% sodium bicarbonate (2×500 mL) and finally with brine (2×500 mL). The organic layer was dried over sodium sulfate and concentrated under reduced pressure to give an oily compound. Yield: 370 g (94%).

(j)Cis/trans-2-(3-Methoxy-4-p-chlorophenylthioethoxy-5-cyanophenyl)-5-(3,4,5-trimethoxyphenyl)tetrahydrofuran (compound 312): In a 3 L round bottom flask, compound 311 (370 g) was dissolved in DMF (900 mL), cuprous cyanide (75.7 g) was then added in one portion, and the reaction mixture was heated to 120-125° C. for 4-5 hours. The reaction was monitored by TLC (silica gel, solvent system: 30% ethyl acetate in n-hexane). The mixture was cooled to room temperature, water (4 L) and benzene (1 L) were added, and the solid was filtered and washed with benzene (500 ML). The organic layer was separated and washed with water (500 mL), brine (2×500 mL), dried over sodium sulfate, and filtered through a silica gel bed. Benzene solution was concentrated under reduced pressure, and the residue used in the next step without further purification. Yield: 230 g (73.4%).

(k) Crystallization of the cis-trans mixture of compound 312 to give pure trans compound 313: The cis-trans mixture of compound 312 (230 g) was dissolved in ethyl acetate (1 L) and n-hexane (900 mL) was slowly added with stirring until turbidity of the solution persisted. The solution was cooled to room temperature, then seeded with pure trans compound, and left standing at −10° C. for 10-12 hours. The white solid was filtered and washed with 20% ethyl acetate in n-hexane four times. The white product was washed with n-hexane (100 mL) and dried under vacuum for 2 hours.

The organic layers were combined and concentrated under reduced pressure. The residue (150 g) was dissolved in chloroform (270 mL) and trifluoroacetic acid (30 mL) was added. The mixture was stirred for 7-8 hours at room temperature. Water (200 mL) was added and the organic layer separated, washed with water (200 mL), 20% sodium bicarbonate solution (200 mL) and finally with brine (200 mL), and dried over sodium sulfate. Chloroform was removed under reduced pressure. The residue was dissolved in ethyl acetate (220 mL) and hexane (500 mL) was added with stirring until turbidity persisted. As above, the solution was seeded with pure trans compound, and left standing at −10° C. for 12-14 hours. The solid was collected by filtration, washed four times with 20% ethyl acetate in n-hexane, and dried under vacuum for 2 hours. The solid thus obtained was thoroughly mixed with the first solid, the mixture suspended in hexane (150 mL), filtered, and dried. Yield: 105 g (45.6%), purity: 97% trans, 1.2% cis, m.p.: 85-86° C.

(l) Trans-Methoxy-4-p-chlorophenylthioethoxy-5-aminomethylphenyl)-5-(3,4,5-trimethoxyphenyl)tetrahydrofuran (compound 314; note—same compound as 110 in Example 1, FIG. 1c): Compound 313 (100 g) was dissolved in THF (500 mL) and cooled to 0° C. in a 2 L round bottom flask. Then alane-N,N-dimethylethylamine complex in toluene (0.5 M, 800 mL) was slowly added under a N₂ atmosphere. The reaction mixture was then refluxed for 2 hours, stirred at room temperature for 1 hour, and then cooled to 0° C. The reaction was quenched with saturated sodium chloride solution (50 mL), the solid collected by filtration, and washed with hot THF (2×100 mL). The combined filtrate and washings were concentrated under reduced pressure. To the residue obtained, toluene (100 mL) was added and then removed under reduced pressure to give a thick oil. Yield: 95.6 g (95%).

(m) CMI-392, (±) trans-2-[5-(N′-methyl-N′-hydroxyureidylmethyl)-3-methoxy-4-p-chlorophenylthioethoxyphenyl]-5-(3,4,5-trimethoxyphenyl)tetrahydrofuran: To a 2 L round bottom flask containing toluene (400 mL) cooled to 0° C. was added p-nitrophenylchloroformate (36 g). Then, compound 314 (95 g) dissolved in toluene (400 mL) was slowly added followed by triethylamine (18 g). The reaction mixture was stirred at 0° C. for 2.5 hours. In a separate flask, to N-methylhydroxylamine HCl (21.3 g) in DCM (200 mL) was added triethylamine (27 g). The resultant mixture was added to the reaction vessel above along with triethylamine (18 g). The reaction mixture was heated at 60-65° C. for 3 hours, and monitored by TLC (silica gel, solvent system: 60% ethyl acetate in hexane). The reaction mixture was cooled to room temperature, water (500 mL) was then added, and the organic layer was separated and washed with 10% potassium hydrogen sulfate (1×300 mL, 2×150 mL), 1N NaOH solution (800 mL), brine (4×250 mL), 10% potassium hydrogen sulfate solution (200 mL) and finally with brine (500 mL). The organic layer was dried over sodium sulfate and concentrated to give an oil. Yield: 105 g (98%).

(n) Purification of CMI-392: The oily product obtained in the preceding step was dissolved in isopropyl alcohol (300 mL) by warming to 45-50° C. The solution was then cooled to −10° C. for 12 hours. To the cold solution, n-hexane (300 mL) was added, seeded with pure CMI-392, and left below −10° C. for another 10-12 hours. The solid was collected by filtration, washed with 5% isopropyl alcohol in n-hexane (100 mL) and dried. The product was recrystallized from isopropyl alcohol in hexane (1:1) as above, and washed with 10% isopropyl alcohol in n-hexane (4×150 mL). The compound was then suspended in n-hexane (100 mL), filtered, and dried under vacuum for 2 hours. Yield: 70 g (67%), purity: 98% (HPLC), m.p.: 54-55° C.

### (B) Using Aspirin as a Starting Material

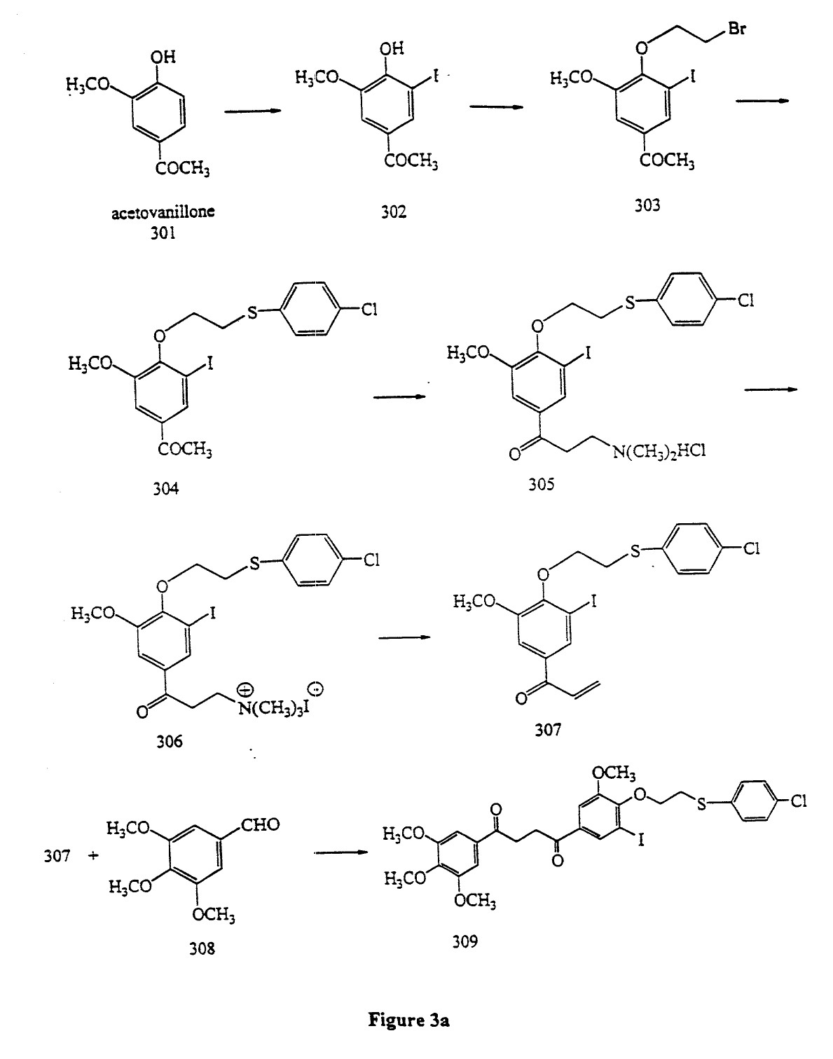

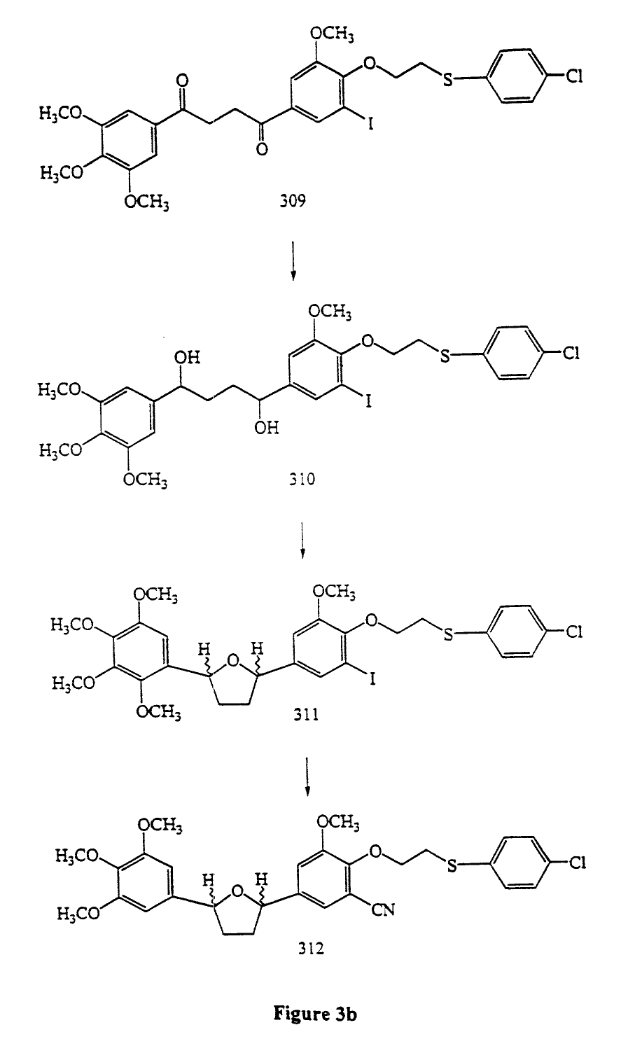

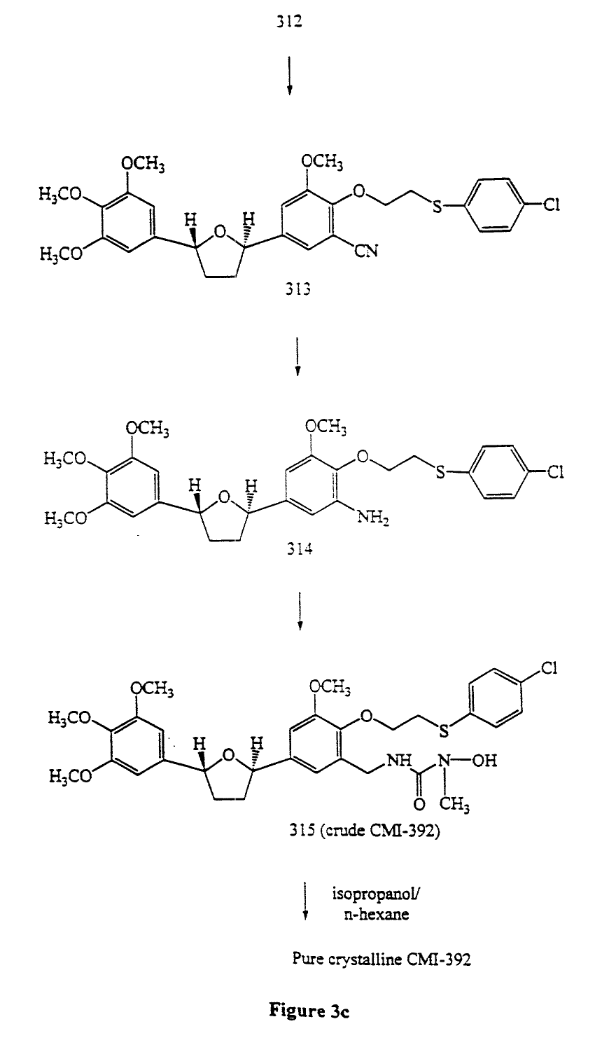

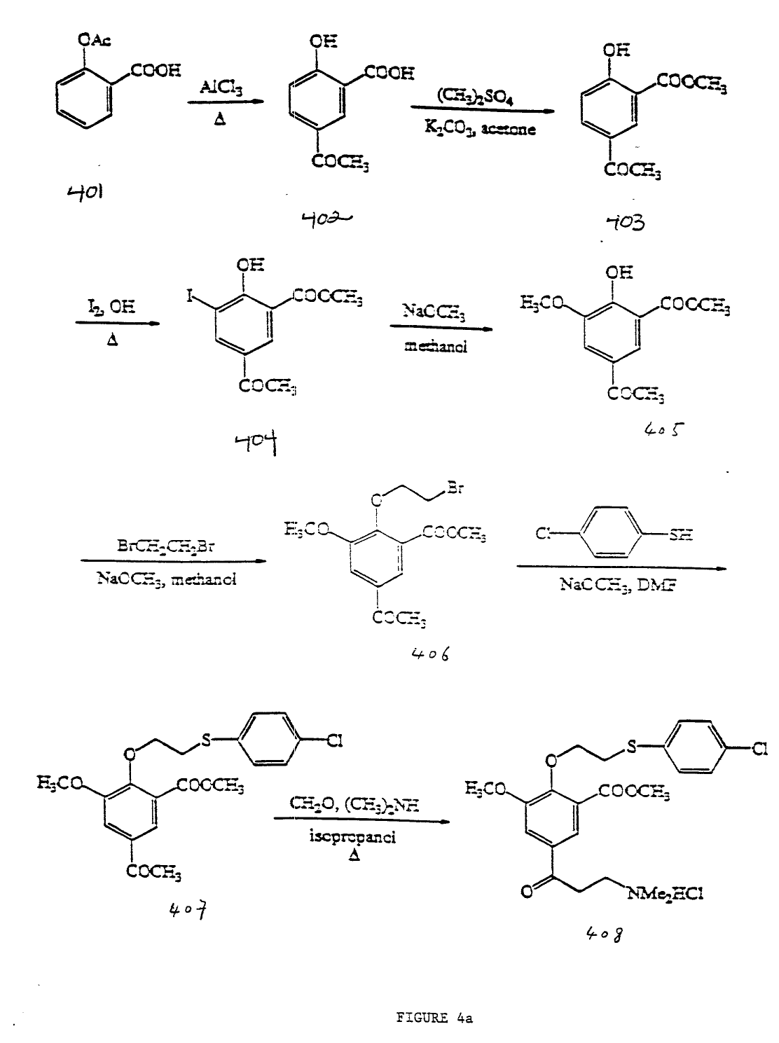

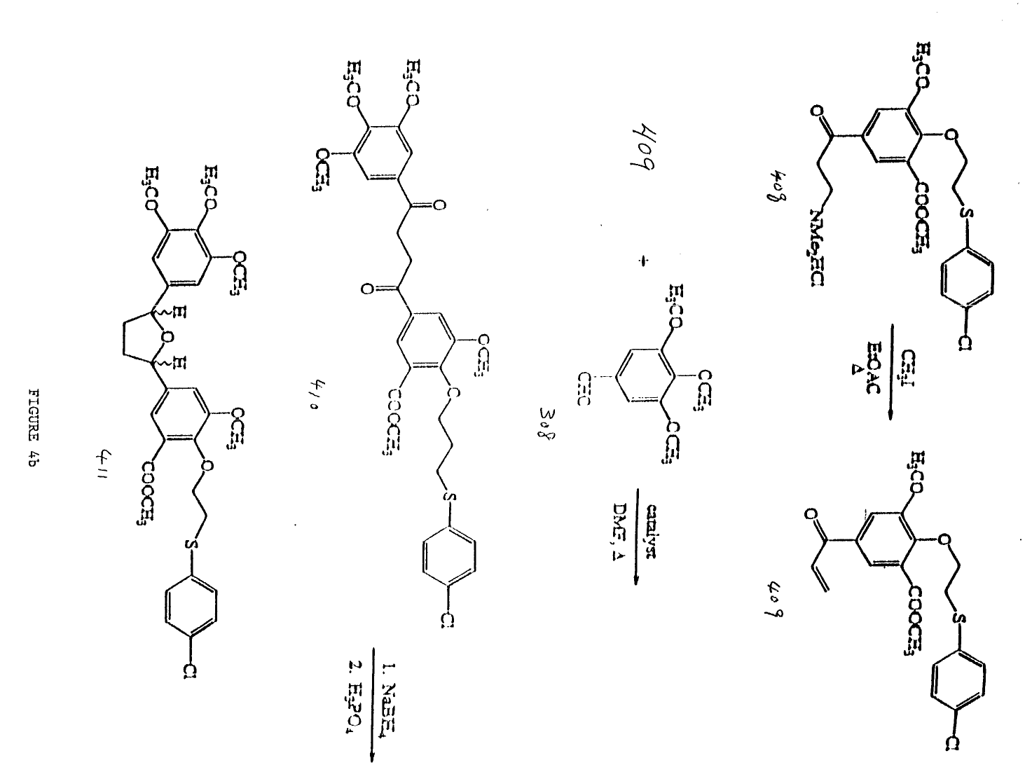

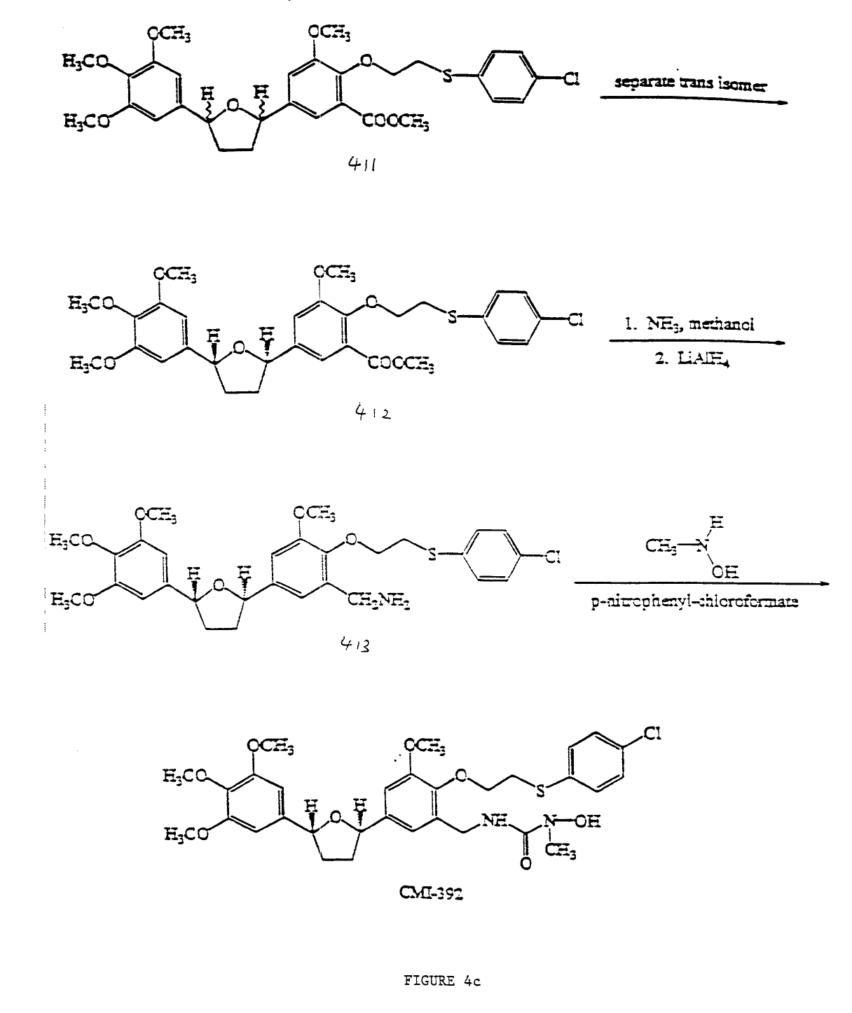

Methyl salicylate (Aspirin, FIG. 4a, 401) was heated with a Friedel-Crafts catalyst such as AICl3 to give Fries rearrangement product (402). Methylation of 402 using dimethyl sulfate in presence of anhydrous potassium carbonate furnished methyl ester (403), which was then reacted with iodine under basic condition to yield 404. Aromatic substitution of the iodide by methoxy group followed by O-alkylation using 1-2 dibromoethane gave 406. Compound 406 was treated with a mild base followed by p-chlorothiophenol to furnish 407, which was then reacted with paraformaldehyde and suitable amine hydrochloride such as dimethylamine HCl, in appropriate solvent such as isopropanol to yield Mannich salt (408) on heating. Compound 408 was converted to the quaternary ammonium salt with methyl iodide which on heating was converted to enone 409. Catalytic coupling of the enone (409) and trimethoxy benzaldehyde (308, see FIG. 3) in DMF furnished diketone 411. Reduction of the diketone (410) with a suitable reducing agent such as NaBH4 followed by acid catalysed cyclization gave substituted tetrabydrofuran, 411. Treatment of the separated bans isomer of the substituted tetrahydrofuran, (412) with ammonia in methanol and subsequent reduction furnished amine 413. The amine (413) on reaction with p-nitrophenyl chloroformate, and N-methylLydroxylamine in the presence of a base such as triethyl amine furnished CMI-392. CMI-392 was then crystallized in isopropyl alcohol.

### EXAMPLE 4

### Scaled-Up Synthesis of CMI-392

(a) 3,4,5-Trimethoxybenzoyl chloride (compound 202): To a 500 L, all glass assembly containing 150 L of benzene, 3,4,5-trimethoxy benzoic acid (100.0 kg) and DMF (2 L) were added. SOCl₂ (73.0 kg) was then added to the solution over a 2-3 hour period, and the contents stirred at room temperature for at least 4 hours. The solution was then heated to 50-60° C. for 1 hour and the benzene along with the excess SOCl₂ was distilled off. The product 202 was crystallized from hexane. The mother liquor was concentrated under reduced pressure, and a second crop was collected. The products from the first and second crystallization were blended thoroughly. Yield: 101 kg (93%/0), m.p.: 78-79° C.

(b) 3,4,5-Trimethoxybenzoyl acetate (compound 203): In a 1000 L reactor, water (300 L) and ethyl acetoacetate (57 kg) were added with vigorous stirring, followed by toluene (100 L) and triethylbenzyl ammonium chloride (500 g) and contents cooled to 0-5° C. To the stirred solution sodium hydroxide (32%, 25 L) was added in 1 hour. The contents were stirred for an additional hour. To this, trimethoxybenzoyl chloride (101 kg) dissolved in hot toluene (250 L, 100-110° C.) and the remaining aqueous NaOH (32%, 100 L) were simultaneously added while maintaining the temperature of the reaction solution below 5° C. and pH between 10-11. The contents were stirred for an additional 2 hours at room temperature. The stirring was then stopped and the two layers separated. The aqueous layer was then heated with NH₄Cl (24 kg) and stirred for 12-14 hours. The solid was then collected by filteration, washed and dried at room temperature. Yield: 62 kg (50.4%), m.p.: 92° C.

(c) 3,4,5-Trimethoxyacetophenone (compound 204): In a 250 L reactor, concentrated sulfuric acid (42 L) was slowly added to water (260 L) followed by the keto ester 203 obtained in the preceding example (30.0 kg). The mixture was stirred at reflux for 6-7 hours. The acidic solution was then allowed to reach room temperature and the solid was collected by filtration. Yield: 20.1 kg (90%), m.p.: 75° C.

(d) 3,4,5-Trimethoxyphenyl-(N,N-dimethylaminoethyl)ketone (compound 205): In a 250 L all glass assembly, compound 204 (20 kg) in isopropanol (80 L), paraformaldehyde (5.8 kg), dimethylamine HCl (14 kg), and concentrated HCl (1.0 L) were combined and reflux-d for 7-8 hours. Half of the isopropanol was distilled off and the contents were cooled. The solid thus obtained was stirred in acetone (40 L) for 2 hours, and then collected by filtration. Yield: 22.5 kg (77.8%), m.p.: 173-175° C.

(e) Quaternary ammonium salt 207: In a 100 L all glass assembly, compound 206 (20 kg) was stirred with 40 L of 10% aqueous NaOH. The free base thus liberated was extracted with ethyl acetate (100 L). The organic layer was collected and cooled to 0-5° C. To this cold solution, methyl iodide (12.2 kg) was added in three equal portions and stirred at room temperature for 8-10 hours. The solid that separated was filtered, washed with ethyl acetate (20 L), and dried under reduced pressure. Yield: 22.5 kg (83.5%), m.p.: 180° C.

(f) 3,4,5-Trimethoxyphenyl vinyl ketone (compound 208): Compound 207 (10 kg) was dissolved in 100 L of water in a 250 L glass assembly, and stirred at 40° C. for 2 hours. To this was added ethyl acetate (60 L) and the contents were refluxed for two hours. The organic layer was collected and the aqueous layer was once again mixed with ethyl acetate (20 L) and refluxed for 1 hour. After separating the organic layer, the aqueous layer was subjected to the same treatment (10 L). All the organic extracts were combined and the solvent was removed under reduced pressure to give an oil which solidified on standing. Yield: 4.1 kg (75.5%/0), m.p.: 42-43° C.

(g) 5-Iodovanillin (compound 210): Vanillin (20 kg) was dissolved in a 6.3% aqueous sodium hydroxide solution (100 L) in a 250 L, all glass assembly, and the solution was heated to 50° C. To this, a first portion of iodine (11 kg) was added over a 0.5 hour period, a second portion of iodine (11 kg) was added over 1 hour period, and a last portion of iodine (12 kg) was added over a 0.5 hour period and the contents heated to 90-95° C. for 3-4 hours. The reaction was monitored by TLC (silica gel, solvent system: benzene). The contents were cooled to room temperature and the solid was collected by filteration. The solid, iodovanillin, was washed with sodium dithionite (5.0 kg dissolved in 50 L water). The dark brown compound was dried in an oven (heated by dry air at 80° C.) for 10-12 hours. The crude dark colored iodovanillin (38.8 kg) was taken in isopropanol (80 L) and heated to 78-82° C. for 3 hours. The contents were then cooled to room temperature, the solid collected by filteration, washed with isopropanol (10 L) and dried in the oven (70-75° C. for 10-2 hours). Yield: 30 kg (82%), m.p.: 179-180 C.

(h) 3-Methoxy-4-bromoethoxy-5-iodovanillin(compound 211): In a 100 L reactor, 5-iodovanillin (10.0 kg) was dissolved in 50.0 L of DMF. Anhydrous K₂CO₃ (7.5 kg) was added, followed by 1,2-dibromoethane (23.6 kg). The mixture was heated to 70-80° C. for a period of 4 hours. The reaction was monitored by TLC (silica gel, solvent system: 10% benzene in hexane). The contents were allowed to cool to room temperature, and the inorganic salts were collected by filtration, and washed with DMF. The combined filtrates were concentrated under reduced pressure below 60° C. to yield an oily product. The oil was dissolved in benzene (50 L) and washed with brine (2×20 L), and the organic solvent removed under reduced pressure to give an oily product. The oily product was then cooled to 0-5° C., methanol (5 L) was added followed by the addition of hexane (25 L) to yield a crystalline product, 211. Yield: 10.5 kg (75.8%), m.p.: 73-75° C.

(i) 3-Methoxy-,4-(p-chlorophenylethylthio)-oxy-5-iodobenzaldehyde (compound 213): Sodium methoxide (2 kg) was added to 20 L THF in a 100 L reactor at 0-5 C. A solution of p-chlorothiophenol dissolved in THF (5 kg in 10 L of THF) was added while the temperature was maintained at 0-5° C., and then stirred at room temperature for another 2 hours. To this was added a solution of compound 211 dissolved in THF (12 kg in 30 L) followed by NH₄Cl (100 g). After stirring for 4-5 hours at room temperature, TLC conformed the absence of starting material IIc (silica gel, solvent system: 10% benzene in n-hexane). The solution was then quenched with a salt solution of NH₄Cl (2.5 kg in 5 L of water) and stirred for 1 hour.

The THF layer was collected and the solvent removed under reduced pressure. The residue was dissolved in dichloromethane (30 L) and the organic layer was washed with a brine solution (2×20 L). Dichloromethane was removed, the oil stirred in n-hexane (12 L) for 1 hour, the hexanes decanted, and methanol (6 L) was added to the oil and cooled to 0° C. The solid was collected by filtration. Yield: 10 kg (71.5%), m.p.: 64-65° C.

(j) 3-Methoxy-4-(p-chlorophenylethylthio)-oxy-5-cyanobenzaldehyde (compound 214): In a 100 L reactor, DMF (25 L), CuCN (3 kg) and compound 213 (10 kg) were heated, while stirring, to 120-125 C for 4-5 hr. The reaction was monitored by TLC (silica gel, solvent system: 30% ethyl acetate in hexane). The contents were cooled to room temperature and filtered through a celite bed. The benzene layer was washed with water (50 L) and then with brine solution (2×40 L). The solvent was then removed to obtain crude cyano compound 214 as a thick oil which was then stirred with 2.5 L of methanol for 3-4 hours at 0-5° C. The separated solid was collected by filteration, washed with 5% methanol in hexanes (5L), and air dried for 5-6 hours. Yield: 5.6 kg (72%), m.p.: 80-81° C.

(k) 1-(3′,4′,5′-Trimethoxyphenyl)-4-[4′-(p-chlorophenylthioethyl)-oxy-5′-cyano-3′-methoxy]phenyl-1,4-dioxobutane (compound 215): Compound 214 (2.0 kg), prepared as described in the preceding section, was dissolved in DMF (3 L) in a 10 L round bottom flask. Catalyst (310 g), a solution of compound 208 (1.4 kg) in DMF (2 L) followed by TEA (872 g) were added at room temperature and the contents stirred for 1.5-2 hours at 70-75° C. The reaction mixture was then cooled to 30-40° C. and dilute HCl (1 L concentrated HCl in 10 L water) was added slowly with vigorous stirring until pH 2-3 was achieved, and the mixture stirred for additional 2 hours. The solid that separated was filtered off and washed with water (5 L) and the crude product was suspended in hot isopropanol (3 L, 60-70° C.), cooled to room temperature and filtered. Crude product was then dissolved in ethyl acetate (4.5 L) while boiling and n-hexane (9 L) was added until turbidity persisted. The contents were then cooled to room temperature and kept standing for 2-3 hr. The solid was filtered, washed with 10% ethyl acetate in petroleum ether (2.0 L) and air dried. Yield: 2.3 kg (70%), m.p.: 113-115° C.

(l) 1-(3′,4′,5′-Trimethoxyphenyl)-4-[4′-(-chlorophenylthioethyl)-oxy-5′-cyano-3′-methoxy]phenyl-1,4-butanediol (compound 216): Compound 215 was dissolved in THF (2.5 kg in 13 L) in a 20 L flask and methanol (1 L) was added. The contents were cooled to 0° C. and NaBH₄ (200 g) was added in 5-10 g portions over a period of 3-4 hours, while maintaining the temperature in the range of 0 to 10° C. Stirring was continued for 2 hours at below 10° C. The reaction mixture was then quenched with water (1 L) and stirred for 2 hours. The organic solvents were removed under reduced pressure and the residue dissolved in benzene (10 L) and washed with water (5 L). The aqueous layer was extracted with benzene (2×1 L) and the combined organic layers were washed with brine solution (2×4 L). The organic layer was dried over sodium sulfate (2 kg) and the inorganic salts were collected by filtration. The compound in the filtrate was used without further purification in the next step.

(m) Trans-2-(3-Methoxy-4-p-chlorophenylthioethoxy-5-cyanophenyl)-5-(3,4,5-trimethoxyphenyl)tetrahydrofuran (compound 217; note—same compound as 110 in Example 1, FIG. 1c): Benzene solution containing compound 216 (15 L), prepared in the preceding step, and orthophosphoric acid (750 mL) were placed in a 20 L three neck round bottom flask and refluxed for 2 hours. The contents were cooled to room temperature and kept standing for 0.5 hr. The upper benzene layer was taken in a separating funnel and washed with water (3×2 L), 20% NaHCO₃ solution (2×5 L) and finally with bromine (2×4 L). The benzene solution was then passed through a short bed of silica gel and the solvent removed under reduced pressure to give an oily compound that was used without further purification in the next step. Yield: 2.4 kg (98.4%, crude).

(n) Crystallization of the cis-trans mixture of compound 217 to give pure trans compound 218: The mixture of cis-trans compound 217 (2.4 kg) obtained in the preceding step was dissolved in ethyl acetate (2.5 L) by warming to 40-50° C. and n-hexane was added slowly with continuous shaking until turbidity of the solution persisted (˜3.75 L). The solution was cooled to room temperature, then seeded with pure trans compound and left standing at −10° C. for a period of 12-14 hours. A white solid separated out which was filtered and washed with 20% ethyl acetate/n-hexane twice and then air dried for 2 hours. Yield: 820 g, m.p.: 85-86° C., Purity: 97.21% trans, 1.05% cis (HPLC). The filtrate was concentrated and the product was left for isomerization.

(o) Isomerization of cis to trans (compound 218): Trifluoroacetic acid (300 mL) was added to the concentrated mixture of cis-trans compound 217 (1.5 kg; obtained in step (m)) in chloroform (2.7 L) in a 3 L three neck round bottom flask. The mixture was stirred for 7-8 hours at room temperature. Water (2.0 L) was added and the organic layer was separated, washed with 20% NaHCO₃ solution (2×4 L), and finally with brine (2×2 L), and dried over sodium sulfate and filtered. Chloroform was removed under reduced pressure to give and oil that was dissolved in benzene (5 L) and passed through a short bed of silica gel (500 g). The benzene solution was concentrated under reduced pressure and the residue crystallized as in step (m) to obtain a white crystalline solid. Yield: 460 g (96.8% pure trans), m.p. 84-85° C. This procedure was repeated to collect maximum trans isomer (218). Total yield: 1.225 kg (98.0% trans), m.p. 85-86 C.

(p) Trans-2-(3′,4′,5′-trimethoxyphenyl)-5-[(p-chlorophenylthioethyl)-oxy-5′-aminomethyl-3′-methoxy]tetrahydrofuran (compound 219): Toluene (8 L) in a 20 L three neck round bottom flask was cooled to 0-5° C. to which lithium aluminum hydride (304 g) was added over a 1 hour period. N,N-dimethylamine was added to the lithium aluminum hydride suspension, and the resultant mixture was stirred for 20-22 hours with gradual warming to room temperature. The reaction mixture was kept undisturbed for another 24 hours and the upper clear solution (8 L) was decanted under nitrogen atmosphere. In a second 20 L three neck flask were placed compound 218 (1.2 kg) in THF (6 L) and cooled to 0° C. The N,N-dimethylethyl-amine complex prepared in the preceding step is then added slowly during a 1 hour period, and the solution refluxed for 1.5-2 hours. The reaction was monitored using TLC (silica gel, solvent system: ethyl acetate and acetone 1:1). The contents were then cooled to 0° C., stirred for an additional hour, and quenched with saturated sodium sulfate solution (250 ml). The inorganic salts were filtered off, extracted with hot tetrahydrofuran (2×2 L) and again filtered. The combined filtrates were concentrated, the residue was dissolved in toluene (1 L), and the organic layer was removed under reduced pressure to give a thick oil. Yield: 1.25 kg.

(q) CMI-392, (±) trans-2-[5-(N′-methyl-N′-hydroxyureidylmethyl)-3-methoxy-4-p-chlorophenylthioethoxyphenyl]-5-(3,4,5-trimethoxyphenyl)tetrahydrofuran: To a 20 L three neck round bottom flask containing toluene (4.5 L) cooled to 0° C. was added p-nitrophenyl-chloroformate (435 g) and then compound 219 (1.13 kg) dissolved in toluene (4.5 L). Then triethylamine (220 g) was added to the reaction mixture. The mixture was stirred for 3 hr at room temperature under nitrogen. The reaction was monitored by TLC (silica gel, solvent system: ethyl acetate and acetone 1:1). In a separate conical flask, triethylamine (350 g) was added to N-methylhydroxylamine HCl (275 g) in dichloromethane (4.5 L). The mixture was then added to the solution containing compound 219 and additional triethylamine (220 g) was added, followed by heating to 60-65° C. for a 2-2.5 hour period. The reaction mixture was cooled to room temperature, water (3 L) was then added and the organic layer was separated and washed with 10% potassium hydrogensulfate (2×4 L), brine (4 L), 1N NaOH solution (10 L), brine (4×2L), 10% potassium hydrogensulfate (4 L), and finally brine (4 L). The organic layer was dried over Na₂SO₄ (1 kg) and concentrated to give an oil. Yield: 1.3 kg.

(r) Purification of CMI-392: The oily product obtained in the preceding step was dissolved in isopropyl alcohol (3.9 L) by warming to 45-50° C. and left at below 0° C. for 15 hr. n-Hexane (3.9 L) was added to the chilled solution of CMI-392, seeded with pure CMI-392, and left below 0° C. for another 12-14 hours. The solid was collected by filtration, washed with 5% isopropyl alcohol in n-hexane (1 L) and dried in vacuo for 2 hours. The product was recrystallized again from isopropyl alcohol and n-hexane, and washed with 10% isopropyl alcohol in n-hexane (3×2 L). Yield: 1.02 kg (75%), HPLC >98% pure, m.p.: 55-56° C.

### EXAMPLE 5

### Stability in Isopropanol-Containing Drug Substance

The effect of isopropanol on drug substance stability was evaluated by preparing a composition containing crystalline CMI-392 and 5 wt. % isopropanol (composition no. 28) and evaluating stability over time relative to the same composition containing no isopropanol (composition no. 27). Results are set forth in Table 1:

TABLE 1

| | IPA | | Amount of CMI-392 in % by weight (Percent of T = 0) | | | | | |
|---|---|---|---|---|---|---|---|---|
| Composition | (%) | Temp. (° C.) | T = 0 | T = 2 wk | T = 1 mo | T = 2 mo | T = 3 mo | T = 6 mo |
| 27 | 0 | 25 | 97.0 | 96.3 | 96.1 | 97.3 | 97.0 | 95.8 |
| | | | | (99.3) | (99.1) | (100.3) | (100.0) | (98.8) |
| 27 | 0 | 40 | NA | 95.9 | 94.3 | 78.5 | 73.8 | ND |
| | | | | (98.9) | (97.2) | (80.9) | (76.1) | |
| 28 | 5.0 | 25 | 93.2 | 92.0 | 93.3 | 91.4 | 93.6 | 94.8 |
| | | | | (98.7) | (100.1) | (98.1) | (100.4) | (101.7) |
| 28 | 5.0 | 40 | NA | 91.9 | 91.6 | 91.1 | ND | ND |
| | | | | (98.6) | (98.3) | (97.7) | | |

NA = not applicable
ND = not determined

The samples were filled into amber vials under an argon headspace. The total amounts of CMI-392 were not adjusted by the contents of IPA in each test sample.

### EXAMPLE 6

### Solution Stability Studies

Solutions of crystalline CMI-392 were prepared in a variety of solvents and at varying concentrations. Stability at 40° C./75% relative humidity over time was evaluated, and results are set forth in Table 2:

TABLE 2

| | Sample | | | | | | | | | | |
|---|---|---|---|---|---|---|---|---|---|---|---|
| | Batch | | | | | | % of | | % of | | % of |
| | Number | | | CMI-392 | % of | CMI-392 | Initial | CMI-392 | Initial | CMI-392 | Initial |
| | CMI- | | Theo. % | Assay | Theo. | Assay, | CMI- | Assay, % | CMI- | Assay, % | CMI- |
| | 392 | | of CMI- | Initial | CMI-392 | % w/w @ | 392 @ 2 | w/w @ 1 | 392 @ | w/w @ | 392 @ |
| No | Solution | Solvent(s) | 392 | (% w/w) | Initial | 2 wks | wks. | mo. | 1 mo. | 2 mo. | 2 mo. |
| A | 0.1% | Diisopropyl | 0.0907 | 0.0904 | 99.7 | 0.0861 | 95.2 | 0.082 | 90.7 | NT | NT |
| | 2CM- | adipate, | | | | | | | | | |
| | 131A | CTFA | | | | | | | | | |
| B | 0.05% | Isopropyl | 0.0463 | 0.0330 | 7.13 | 0.0283 | 85.8 | 0.0255 | 77.3 | NT | NT |
| | 2CM- | myristate, | | | | | | | | | |
| | 133B | NF | | | | | | | | | |
| C | 0.05% | Propylene | 0.0491 | 0.0371 | 75.6 | 0.0338 | 91.1 | 0.0297 | 80.1 | NT | NT |
| | 2CM- | Glycol, USP | | | | | | | | | |
| | 134B | | | | | | | | | | |
| D | 0.1% | Alcohol, | 0.0907 | 0.0879 | 96.9 | 0.0821 | 93.4 | 0.0786 | 89.4 | NT | NT |
| | 2CM- | USP | | | | | | | | | |
| | 135A | | | | | | | | | | |
| E | 0.1% | Purified | 0.0907 | 0.0913 | 100.7 | 0.0855 | 93.6 | NT | NT | NT | NT |
| | 2CM- | Water, USP/ | | | | | | | | | |
| | 136A | Alcohol, | | | | | | | | | |
| | | USP (5:95) | | | | | | | | | |
| F | 0.1% | Purified | 0.0907 | 0.0882 | 97.2 | 0.0800 | 90.7 | NT | NT | NT | NT |
| | 2CM- | Water, USP/ | | | | | | | | | |
| | 136B | Alcohol, | | | | | | | | | |
| | | USP (25:75) | | | | | | | | | |
| G | 0.1% | Purified | 0.0907 | 0.0840 | 92.6 | 0.0680 | 81.0 | NT | NT | NT | NT |
| | 2CM- | Water, USP/ | | | | | | | | | |
| | 136C | Alcohol, | | | | | | | | | |
| | | (50:50) | | | | | | | | | |
| H | 0.1% | Benzyl | 0.0907 | 0.0867 | 95.6 | 0.0582 | 67.1 | NT | NT | NT | NT |
| | 2CM- | Alcohol, | | | | | | | | | |
| | 137A | USP | | | | | | | | | |
| I | 0.1% | Propylene | 0.0907 | 0.0873 | 96.3 | 0.0807 | 92.4 | 0.0682 | 78.1 | NT | NT |
| | 2CM- | Carbonate, | | | | | | | | | |
| | 138A | NF | | | | | | | | | |
| J | 0.05% | Mineral Oil, | 0.0479 | ND | NT | NT | NT | NT | NT | NT | NT |
| | 2CM- | USP | | | | | | | | | |
| | 140B | | | | | | | | | | |
| K | 0.1% | Polyethyl- | 0.0907 | 0.0716 | 78.9 | 0.0050 | 7.0 | NT | NT | NT | NT |
| | 2CM- | ene Glycol - | | | | | | | | | |
| | 141A | 400, USP | | | | | | | | | |
| L | 0.1% | Transcutol | 0.0907 | 0.0634 | 69.9 | 0.0040 | 6.3 | NT | NT | NT | NT |
| | 2CM- | (Ethoxydi- | | | | | | | | | |
| | 142A | glycol) | | | | | | | | | |
| M | 0.1% | Diethyl | 0.0907 | 0.0898 | 99.0 | 0.0861 | 95.9 | 0.0806 | 89.8 | NT | NT |
| | 2CM- | Succinate | | | | | | | | | |
| | 143A | | | | | | | | | | |
| N | 0.0995 | Isopropyl | 0.0881 | 0.0926 | 105.1 | 0.0937 | 101.2 | 0.0904 | 97.5 | 0.0896 | 96.8 |
| | % 2CM- | Alcohol, NF | | | | | | | | (0.0871 | (94.1 |
| | 146A | | | | | | | | | @ 3 mo | @ 3 |
| | | | | | | | | | | | mo) |
| O | 0.1% | Miglyol | 0.0885 | 0.0561 | 63.4 | 0.0537 | 95.7 | 0.0507 | 90.4 | 0.0471 | 84.0 |
| | 2CM- | 812, | | | | | | | | | |
| | 144A | GRAS/EP | | | | | | | | | |
| P | 0.1% | Triacetin | 0.0885 | 0.0912 | 103.1 | 0.0896 | 98.2 | 0.0867 | 95.1 | 0.0821 | 90.0 |
| | 2CM- | | | | | | | | | | |
| | 145A | | | | | | | | | | |
| Q | 0.1% | Isopropyl | 0.0885 | 0.0951 | 107.5 | 0.0916 | 96.3 | 0.0848 | 89.2 | 0.0801 | 84 2 |
| | 2CM-14 | Acetate | | | | | | | | | |
| | 7A | | | | | | | | | | |

NT = Not Tested

### EXAMPLE 7

### Ointment Preparation

An ointment formulation (composition no. 1 herein) containing 6 wt. % crystalline CMI-392 was prepared containing the following components:

| | |
|---|---|
| CMI-392 | 6.0 wt. % |
| Diisopropyl adipate, CTFA | 12.0 wt. % |
| Propylene carbonate, NF | 2.0 wt. % |
| Glyceryl monostearate, EP | 3.5 wt. % |
| Butylated hydroxytoluene, NF | 0.05 wt. % |
| Microcrystalline wax, NF | 5.0 wt. % |
| White Petrolatum, USP | QSAD 100.0 |

The white petrolatum, microcrystalline wax, and glyceryl monostearate were weighed into a stainless steel vessel. The components were warmed until complete melt was achieved (70° C. to −15° C.). The mixture was then cooled to approximately 55° C. to 60° C., while stirring at low speed. Diisopropyl adipate and propylene carbonate were weighed out in a stainless steel vessel, butylated hydroxytoluene was added, and the mixture was stirred until solution was achieved. CMI-392 was added to the solution, and the mixture was warmed to between approximately 35° C. and 45° C. to ensure complete dissolution. The CMI-392 solution was then combined with the petrolatum mixture, heated to approximately 60° C. and mixed for at least about five minutes. The mixture was then cooled, with continued mixing, to between about 25° C. and 30° C.

The ointment formulation so prepared has a shelf life on the order of eighteen months at room temperature.

### EXAMPLE 8

### Skin Penetration and Stability Studies: CMI-392 Ointments

CMI-392 ointments (compositions No. 3-8) were prepared using the method described in Example 7, containing components as indicated below:

| | |
|---|---|
| Composition No. 2: | |
| CMI-392 | 6.0 wt. % |
| Acetone | QSAD 100.0 |
| Composition No. 3: | |
| CMI-392 | 6.0 wt. % |
| Diisopropyl adipate | 13.0 wt. % |
| Glyceryl monostearate | 3.5 wt. % |
| Butylated hydroxytoluene | 0.1 wt. % |
| Microcrystalline wax | 5.0 wt. % |
| White petrolatum | QSAD 100.0 |
| Composition No. 4: | |
| CMI-392 | 6.0 wt. % |
| Diisopropyl adipate | 13.0 wt. % |
| Lecithin | 3.5 wt. % |
| Microcrystalline wax | 5.0 wt. % |
| White petrolatum | QSAD 100.0 |
| Composition No. 5: | |
| CMI-392 | 6.0 wt. % |
| Propylene carbonate | 13.0 wt. % |
| Lecithin | 3.5 wt. % |
| Microcrystalline wax | 5.0 wt. % |
| White petrolatum | QSAD 100.0 |
| Composition No. 6: | |
| CMI-392 | 6.0 wt. % |
| Diisopropyl adipate | 6.5 wt. % |
| Propylene carbonate | 6.5 wt. % |
| Lecithin | 3.5 wt. % |
| Microcrystalline wax | 5.0 wt. % |
| White petrolatum | QSAD 100.0 |
| Composition No. 7: | |
| CMI-392 | 6.0 wt. % |
| Diisopropyl adipate | 13.0 wt. % |
| Microcrystalline wax | 5.0 wt. % |
| White petrolatum | QSAD 100.0 |
| Composition No. 8: | |
| CMI-392 | 6.0 wt. % |
| Diisopropyl adipate | 12.0 wt. % |
| Triacetin | 5.0 wt. % |
| Glyceryl monostearate | 3.5 wt. % |
| Microcrystalline wax | 3.0 wt. % |
| White petrolatum | QSAD 100.0 |

Skin penetration was evaluated as follows. Formulations were tested on freshly excised skin obtained from a single pig. The formulations were spiked with C¹⁴-CMI-392 and penetration was assessed by liquid scintillation counting (LSC). Following euthanasia, skin was harvested from the upper back of Yorkshire pigs approximately 40 kg in weight. Excised pig skin was cut with a dermatome to yield a thickness of 0.7 mm, with a range of 0.5-0.9 mm. This “split-thickness” skin was composed of the epidermis and the outer most portion of the dermis containing the papillary dermis. Samples of split thickness skin were attached to penetration cells. The visceral side of the skin sample was bathed by tissue culture medium which served as a sink for radiolabel that penetrated the skin. Different formulations containing the radiolabeled test compound CMI-392 were topically applied in order to obtain three replicates for each of the formulations. Six hours following application, receptor fluid was replaced. Twenty-four hours after application, receptor fluid was collected. The skin surface was decontaminated with a dry cotton ball. The stratum corneum was harvested by tape stripping. Tape strips 1 and 2 were combined in one LSC vial and represented a portion of the skin surface residue of the CMI-392 formulations. Strips 3-22 were counted as the stratum corneum. The remaining epidermis was separated from the dermis. The cotton ball, tape strips, epidermis, dermis and receptor fluid were analyzed for radioactivity. Skin penetration results for acetone (reference; 2) and ointment compositions 3 through 7, above, are set forth in Table 3:

TABLE 3

| | Composition No. | | | | | |
|---|---|---|---|---|---|---|
| | 2 | 3 | 4 | 5 | 6 | 7 |
| Stratum corneum, % | 24.7 | 4.2 | 8.8 | 4.50 | 4.34 | 1.47 |
| Epidermis, T | 4.0 | 0.8 | 1.0 | 1.0 | 0.68 | 0.50 |
| Dermis, % | 0.8 | 0.09 | 0.08 | 0.07 | 0.13 | 0.07 |
| Epidermis + dermis | 4.8 | 0.89 | 1.08 | 1.07 | 0.81 | 0.57 |

Formulation stability was also evaluated; results are set forth in Table 4:

TABLE 4

| Storage Time and | Composition No. | | | | |
|---|---|---|---|---|---|
| Temperature | 3 | 4 | 5 | 6 | 7 |
| Initial | 95.7 | 104.5 | 96.0 | 103.0 | 101.7 |
| 2 weeks, 30° C. | 103.0 | 108.8 | 99.5 | 102.8 | 115.7 |
| 2 weeks, 40° C. | 114.0 | 118.3 | 104.0 | 102.8 | 123.3 |
| 1 month, 30° C. | 105.2 | 105.5 | 99.8 | 105.7 | 131.5 |
| 1 month, 40° C. | 130.7 | 121.8 | 110.3 | 79.0 | 104.7 |
| 2 months, 30° C. | 101.5 | 124.7 | 101.0 | 112.3 | 111.7 |
| 2 months, 40° C. | 134.4 | 123.3 | 105.3 | 119.0 | 137.5 |
| 3 months, 30° C. | — | 101.3 | 94.2 | 94.0 | — |
| 3 months, 40° C. | — | 117.3 | 73.3 | 83.8 | — |

Results are indicated as the percentage of CMI-392 remaining, i.e., the % of the original quantity of 6.0 wt. %.

### EXAMPLE 9

### Cream Preparation

A topical pharmaceutical formulation (composition no. 9 herein) comprising a 5 wt. % crystalline CMI-392 cream was prepared containing the following components:

| | |
|---|---|
| CMI-392 | 5.0 wt. % |
| White soft paraffin | 32.0 wt. % |
| Liquid paraffin | 3.0 wt. % |
| Cetostearyl alcohol | 9.0 wt. % |
| Polysorbate 40 | 8.0 wt. % |
| Glyceryl monostearate | 3.0 wt. % |
| Propylene glycol | 5.0 wt. % |
| Saturated neutral oil | 2.0 wt. % |
| Silicic acid | 0.2 wt. % |
| Sorbic Acid | 0.2 wt. % |
| Water | QSAD 100.0 |

### EXAMPLE 10

### Stability Studies: CMI-392 Cream Formulations

CMI-392 cream formulations were prepared as in Example 9, except that a first formulation (composition no. 10 in Table 5) contained 4.4 wt. % drug, and the second formulation (composition no. 11 in Table 5) contained 8.3 wt. % drug. Stability was evaluated over a two-month period; results are summarized in the following table:

TABLE 5

| Compo- | Concen- | | | | | |
|---|---|---|---|---|---|---|
| sition | tration | Temperature | Amount (% of label) | | | |
| No. | (w/w) | (° C.) | Initial | 0.5-month | 1-month | 2-month |
| 10 | 4.4% | 4 | NA | 92.6 | 109.3 | 98.5 |
| | | 25 | 102.4 | 91.8 | 99.6 | 93.3 |
| | | 40 | NA | 78.6 | 67.3 | 49.2 |
| 11 | 8.3% | 4 | NA | 90.4 | 90.5 | 88.7 |
| | | 25 | 95.9 | 88.2 | 87.3 | 84.3 |
| | | 40 | NA | 81.7 | 68.2 | 61.7 |

The cream formulation prepared by mixing the above components has a shelf life on the order of two months at room temperature.

### EXAMPLE 11

### Additional CMI-392 Creams

Additional CMI-392 creams were prepared using the method described in Example 9. Concentrations of the various components were optimized to provide stable formulations, and optimal composition ranges are set forth in Table 6:

TABLE 6

| | Optimal Concentration Ranges % w/w | | |
|---|---|---|---|
| | Composition | Composition | Composition |
| Component | No. 12 | No. 13 | No. 14 |
| CMI-392 | 3.0 | 3.0 | 5.0-6.0 |
| Diisopropyl adipate, CTFA | 10.0 | 10.0 | 12.0-13.0 |
| Stearyl alcohol, NF | 6.0 | 6.0 | — |
| Sodium lauryl sulfate, NF | 0-0.5 | — | — |
| Povidone 90, USP | 0-3.0 | 0-3.0 | — |
| Methylparaben, NF | 0.15 | 0.15 | — |
| Propylparaben, NF | 0.03 | 0.03 | — |
| Butylated hydroxytoluene, | 0-0.05 | — | — |
| NF | | | |
| Trolamine (50%), NF | 0.10-0.15 | 0.10 | — |
| Carbomer 1342, NF | 0.15-0.20 | 0.10-0.15 | 1.0 |
| Brij 72 (polyoxyethylene 2 | — | 1.50 | — |
| stearyl ether) | | | |
| Brij 721 (polyoxyethylene | — | 1.50 | — |
| 21 stearyl ether) | | | |
| Isopropyl alcohol, USP | — | 0-10.0 | — |
| Cyclodextrin, NF | 0-0.30 | 0-0.30 | — |
| White Petrolatum, USP | 6.0 | 6.0-20.0 | 0-5.0 |
| Potassium Sorbate, NF | — | — | 0.2 |
| Sodium Dehydroacetate, NF | — | — | 0.2 |
| Aqueous Lecithin, NF | — | — | 0-0.5 |
| Sodium Hydroxide, 10% | — | — | qs pH 4.5 |
| Purified water, USP | QSAD 100.0 | QSAD 100.0 | QSAD 100.0 |

Additional methods of preparing CMI-392 creams were conducted according to the following method:

Method of preparation of cream formula: Add CMI-392 to Diisopropyl Adipate and warm to about 40° C. with stirring to dissolve. Combine remainder of oil phase components as present (Stearyl Alcohol, Brij 72, Brij 721, White Petrolatum, BHT) in separate vessel and warm to melt completely. In a separate vessel warm the Purified Water to about 70° C. Add the preservatives and aqueous phase additives as present (Methylparaben, Propylparaben, Potassium Sorbate, Sodium Dehydroacetate, Povidone 90, Cyclodextrin, Sodium Lauryl Sulfate, Lecithin) and stir to dissolve. Add the carbopol 1342 to aqueous mixture and stir to disperse. With high-speed stirring add the melted oil phase and the CMI-392 solution to aqueous phase and stir to form homogeneous emulsion. Add Trolamine (50%) or Sodium Hydroxide (10%) to neutralize. Add Isopropyl Alcohol as present and stir well. Cool emulsion to room temperature with stirring.

### EXAMPLE 12

### CMI-392 Solutions

Extended stability studies were conducted with three solution formulations containing crystalline CMI-392, with the compositions and results as follows:

TABLE 7

| | Composition | Composition | Composition |
|---|---|---|---|
| | 15 | 16 | 17 |
| CMI-392 | 6.0 wt. % | 6.0 wt. % | 6.0 wt. % |
| Triacetin | — | — | 15.0 wt. % |
| Diisopropyl adipate, CTFA | 20.0 wt. % | 22.0 wt. % | 15.0 wt. % |
| Propylene carbonate, NF | — | 6.0 wt. % | — |
| Diethyl succinate | 20.0 wt. % | — | — |
| Isopropyl alcohol | QSAD 100.0 | QSAD 100.0 | QSAD 100.0 |

Stability Results are found in the following table:

TABLE 8

| | Composition | Composition | Composition |
|---|---|---|---|
| | 15 | 16 | 17 |
| T = 0, % w/w | 5.84 | 5.80 | 5.85 |
| 1 wk: 25° C./60% RH-U, | 5.72 | 5.73 | 5.78 |
| % w,w | | | |
| 1 wk: 25° C./60% RH-I, | 576 | 5.74 | 5.75 |
| % w,w | | | |
| 1 wk: 40° C./75% RH-U, | 5.68 | 5.65 | 5.70 |
| % w,w | | | |
| 1 wk: 40° C./75% RH-I, | 5.69 | 5.69 | 5.67 |
| % w,w | | | |
| 1 wk: Freeze/Thaw-U, | 5.75 | 5.73 | 5.75 |
| % w/w | | | |
| 1 wk: Freeze/Thaw-I, | 5.73 | 5.73 | 5.73 |
| % w/w | | | |
| 1 wk: Cool/Warm-U, | 5.67 | 5.68 | 5.69 |
| % w/w | | | |
| 1 wk: Cool/Warm-I, | 5.66 | 5.70 | 5.71 |
| % w/w | | | |
| 2 wk: 25° C./60% RH-U, | 5.73 | 5.72 | 5.75 |
| % w/w | | | |
| 2 wk: 25° C./60% RH-I, | 5.73 | 5.71 | 5.74 |
| % w/w | | | |
| 2 wk: 40° C./75% RH-U, | 5.62 | 5.65 | 5.66 |
| % w/w | | | |
| 2 wk: 40° C./75% RH-I, | 5.63 | 5.65 | 5.65 |
| % w/w | | | |
| 2 wk: Freeze/Thaw-U, | 5.73 | 5.72 | 5.79 |
| % w/w | | | |
| 2 wk: Freeze/Thaw-I, | 5.77 | 5.74 | 5.76 |
| % w/w | | | |
| 2 wk: Cool/Warm-U, | 5.66 | 5.66 | 5.66 |
| % w/w | | | |
| 2 wk: Cool/Warm-I, | 5.69 | 5.67 | 5.66 |
| % w/w | | | |
| 4 wk: 25° C./60% RH-U, | 5.74 | 5.71 | 5.67 |
| % w/w | | | |
| 4 wk: 25° C./60% RH-I, | 575 | 5.70 | 5.70 |
| % w/w | | | |
| 4 wk: 40° C./75% RH-U, | 5.58 | 5.59 | 5.62 |
| % w/w | | | |
| 4 wk: 40° C./75% RH-I, | 5.55 | 5.56 | 5.55 |
| % w/w | | | |
| 4 wk: Freeze/Thaw-U, | 5.70 | 5.70 | 5.75 |
| % w/w | | | |
| 4 wk: Freeze/Thaw-I, | 5.71 | 5.68 | 5.72 |
| % w/w | | | |
| 4 wk: Cool/Warm-U, | 5.63 | 5.59 | 5.69 |
| % w/w | | | |
| 4 wk: Cool/Warm-I, | 5.59 | 5.54 | 5.63 |
| % w/w | | | |
| 2 mo: 25° C./60% RH-U, | 5.65 | 5.65 | 5.72 |
| % w/w | | | |
| 2 mo: 25° C./60% RH-I, | 5.66 | 5.67 | 5.71 |
| % w/w | | | |
| 2 mo: 40° C./75% RH-U, | 5.42 | 5.38 | 5.50 |
| % w/w | | | |
| 2 mo: 40° C./75% RH-I, | 5.41 | 5.42 | 5.47 |
| % w/w | | | |
| 3 mo: 25° C./60% RH-U, | 5.59 | 5.63 | 5.43 |
| % w/w | | | |
| 3 mo: 25° C./60% RH-I, | 5.62 | 5.64 | 5.61 |
| % w/w | | | |
| 3 mo: 40° C./75% RH-U, | 5.13 | 5.13 | 5.27 |
| % w/w | | | |
| 3 mo: 40° C./75% RH-I, | 5.23 | 5.10 | 5.27 |
| % w/w | | | |

Key: U = vial stored upright;
I = vial stored inverted;
RH = relative humidity. EXAMPLE 13

### EXAMPLE 13

### CMI-392 Oils

Oil formulations containing crystalline CMI-392 were prepared containing the following components:

TABLE 9

| | Composition | Composition | Composition |
|---|---|---|---|
| | 18 | 19 | 20 |
| CMI-392 | 6.0 | 6.0 | 6.0 |
| Diisopropyl Adipate, CTFA | — | — | 6.5 |
| Triacetin, USP | 36.0 | 36.0 | 20.0 |
| Colloidal Silicon Dioxide, NF | — | 5.0 | — |
| Miglyol 812, GRAS/EP | QSAD 100.0 | QSAD 100.0 | QSAD 100.0 |

Skin penetration tests and stability studies were carried out as described in Example 8. Results are as follows:

Skin Penetration:

TABLE 10

| | Composition | | Composition |
|---|---|---|---|
| | 18 | Composition 19 | 20 |
| Stratum Corneum, % | 27.0 | 12.4 | 21.7 |
| Epidermis, % | 3.0 | 1.6 | 2.43 |
| Dermis, % | 1.7 | 0.6 | 2.0 |
| E + D, % | 4.7 | 2.2 | 4.43 |

Stability Results, CMI-392, % of “Label Claim” (6.0%):

TABLE 11

| | | Composition | | |
|---|---|---|---|---|
| | | 18 | Composition 19 | Composition 20 |
| Initial | | 98.8 | 101.5 | 101.3 |
| 2 Week | 30° C. | 102.8 | 94.7 | 89.2 |
| | 40° C. | 95.0 | 92.5 | 90.7 |
| 1 Month | 30° C. | 89.3 | NT | 80.5 |
| | 40° C. | 88.8 | NT | 76.7 |
| 2 Months | 30° C. | 91.5 | NT | 78.7 |
| | 40° C. | 81.5 | NT | 69.8 |

NT = Not Tested

### EXAMPLE 14

### Additional CMI-392 Oil Compositions and Stability Data Summary

CMI-392 oil compositions were prepared containing the following components.

TABLE 12

| Composition No. | 1 | 2 | 3 | 4 | 5 | 6 |
|---|---|---|---|---|---|---|
| Diisopropyl Adipate, CTFA | 12.0 | — | 12.0 | 12.0 | — | 12.0 |
| Triacetin, USP | 12.0 | 35.0 | 12.0 | 12.0 | 35.0 | 12.0 |
| Ethyl Oleate, NF | — | — | 1.0 | — | — | — |
| Isopropyl Alcohol, USP | — | — | — | 10.0 | — | — |
| Miglyol 812¹ | — | — | QSAD | — | QSAD | QSAD |
| | | | 100.0 | | 100.0 | 100.0 |
| Miglyol 840² | QSAD | QSAD | — | QSAD | — | — |
| | 100.0 | 100.0 | | 100.0 | | |

Formulation stability also was evaluated of these oil compositions with results set forth in the following Table. In the Table, results are indicated as the percentage of CMI-392 remaining at the end of the specified time period, i.e. the remaining percentage of the original composition quantity of 6.0 weight percent.

TABLE 13

| Interval, Condition | CMI-392 Assay Result, % w/w | | | | | |
|---|---|---|---|---|---|---|
| Composition No. | 1 | 2 | 3 | 4 | 5 | 6 |
| T = 0 | 5.92 | 6.01 | 5.95 | 6.01 | 4.80 | 5.75 |
| T = 0 (repeat) | 5.99 | 5.97 | 5.17 | Not | 5.80 | 5.32 |
| | | | | tested | | |
| T = 2 weeks, 30° C. | 5.92 | 5.88 | 5.13 | 5.99 | 6.09 | 5.49 |
| T = 2 weeks, 40° C./75% RH | 5.85 | 5.83 | 5.16 | 5.92 | 6.03 | 7.08 |
| T = 4 weeks, 30° C. | 5.81 | 5.80 | 5.06 | 5.93 | 6.09 | 5.30 |
| T = 4 weeks, 40° C./75% RH | 5.66 | 5.62 | 5.04 | 5.80 | 5.61 | 5.05 |
| T = 8 weeks, 30° C. | 5.76 | 5.75 | 5.03 | 5.90 | 5.81 | 5.17 |
| T = 8 weeks, 40° C./75% RH | 5.47 | 5.42 | 4.67 | 5.67 | 5.42 | 4.98 |
| T = 12 weeks, 30° C. | 5.65 | 5.88 | 4.87 | 5.81 | 5.52 | 4.97 |
| T = 12 weeks, 40° C./ | 5.28 | 5.18 | 4.44 | 5.45 | 5.25 | 4.56 |
| 75% RH | | | | | | |

¹ Caprylic/Capric Triglyceride, CTFA
² Propylene Glycol Dicaprylate/Dicaprate, CTFA

### EXAMPLE 15

### Ocular Administration

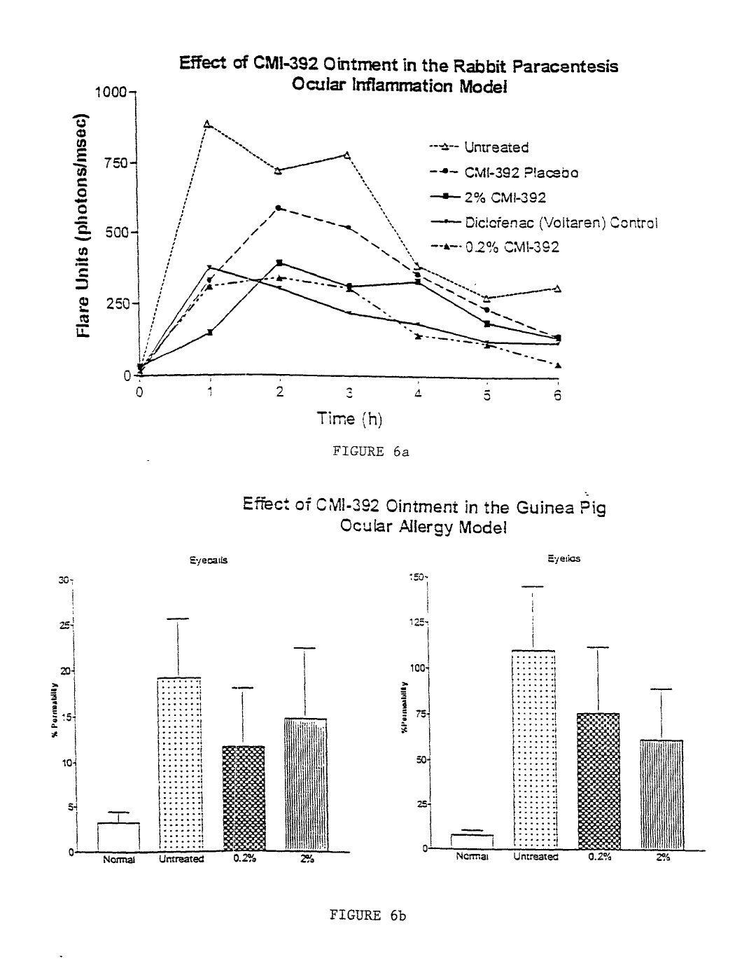

The effects of CMI-392 on a rabbit model of ocular inflammation were assessed. Groups of animals were pretreated with an 0.2% or 2% CMI-392 (in a white petrolatum ointment containing 6.0% diisopropyl adipate and 1.5% glyceryl monostearate) for two hours prior to complete aqueous humor paracentesis. The amount of inflammation was assessed using laser flare meter prior to paracentesis, and at hourly intervals for six hours post-dosing. Additionally, a group of animals was treated with 0.1% diclofenac (Voltaren) as a positive control. Both 0.2% and 2% ointments were equally effective as diclofenac in inhibiting the inflammatory reaction in the eye. Results are set forth in FIG. 6a of the drawings.

The ability of the CMI-392 ointment to inhibit ocular inflammation in an allergy model also was assessed. Groups of guinea pigs were immunized with ovalbumin and then an ocular challenge was given 14 days later. Ointment containing 0.2% or 2% CMI-392, or a positive control agent (0.05% levoptha) were administered to the eyes four times per day for the two days prior to challenge, and five times in the three hours immediately prior to the challenge. The inflammation was measured by assessing the amount of Evans Blues dye extracted from the eyelids and eyeballs 20 minutes after challenge. As shown in FIG. 6b, there was a reduction in the amount of inflammation at both dose levels.

### EXAMPLE 16

### CMI-39:. For the Treatment of Colonic Inflammation

Elevated levels of Platelet activating factor (PAF) and leukotrienes are observed in various inflammatory bowel disease, including Crohn's disease, and ulcerative colitis. There is recognition that that administration of either a PAF receptor antagonist, an inhibitor of leukotriene synthesis, or leukotriene receptor antagonist, is effective in reducing the severity of the disease. This effect was observed in both animal models and in clinical studies.

Experimental data have indicated that inhibiting the action of both LT and PAF results in additive or synergistic reduction in inflammatory responses. CMI-392 is a dual-acting compound designed to inhibit both LT production and antagonize PAF binding: when administered topically or intravenously, CMI-392 exerts significantly higher anti-inflammatory activity than single-acting 5-LO inhibitors or PAF receptor antagonists in a number of animal models.

CMI-392 is an inhibitor of topical arachidonic acid-induced ear edema in the mouse. Arachidonic acid was applied to the mouse ears following i.v. or topical administration of CMI-392, and edema was measured by comparing the average wet weight of the ear punch biopsies obtained from treated and untreated animals. CMI-392 was shown to inhibit arachidonic acid-induced ear edema dose-dependently with an i.v. ED₅₀ of 1.7 mg/kg and a topical ED₅₀ of 0.28 mg/ear.

Topical CMI-392 also resulted in a significant decrease in overall ear mass and inflammatory cell infiltration following both acute and chronic 12-o-tetradecanoyl-phorbol-13-acetate (TPA) application: the ED₅₀ for the acute model was 0.117-0.233 mg/ear, and the ED₅₀ for the chronic model was 0.552 mg/ear. CMI-392 also inhibited U-VB irradiation induced erythema in guinea pigs with an ED₅₀ of 0.92 mg/area.

The effects of CMI-392 on the prevention of colitis has been assessed experimentally using DSS-induced colitis in the mouse. Groups of animals were fed ad libitum with drinking water containing 5% DSS for six days. CMI-392 (at a dose level of 50, 100, 150 or 200 mg/kg/day) in corn oil containing 1% Span 20 was given daily, either by oral gavage or intra-rectally, to groups of mice, starting one day prior to DSS exposure. After four days, signs of acute disease occur with weight loss, diarrhea, and bloody stools. Histological changes include initial shortening of the crypts, then areas of separation of the crypts and muscularis mucosae in the absence of inflammatory infiltrate. After five days the pathological changes become confluent with the appearance of erosions and early hyperplastic epithelium. Inflammation scores are high with neutrophils, lymphocytes, and plasma cells in the lamina propria but sparing the epithelium.

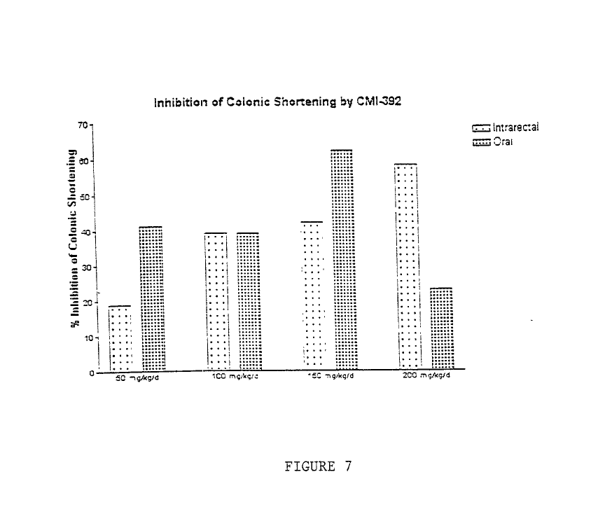

There was a significant dose-related decrease in the severity of the colitis for each group treated with CMI-392, as illustrated in FIG. 7 by the reduced colonic shortening. This indicates that CMI-392 may have a protective effect on the formation of colitis in the DSS-induced colitis, model.

What is claimed is:

1. A topical pharmaceutical formulation for treating inflammatory and immune disorders, comprising, in an ointment base, a therapeutically effective amount of an active agent selected from the group consisting of 2,5-diaryl tetrahydrofurans, 2,5-diaryl tetrahydro-thiophenes, 2,4-diaryl tetrahydrofurans, 2,4-diaryl tetrahydrothiophenes, 1,3-diaryl cyclopentanes, 2,4-diaryl pyrrolidines, and 2,5-diaryl pyrrolidines, and an effective amount of an enhancer composition comprising at least one C₃-C₁₈ saturated ester containing one to three ester functionalities.

2. The formulation of claim 1, wherein the active agent is a 2,5-diaryl tetrahydrofuran, a 2,5-diaryl tetrahydrothiophene, a 2,5-diaryl pyrrolidine, or a 1,3-diaryl cyclopentane.

3. The formulation of claim 2, wherein the active agent is a 2,5-diaryl tetrahydrofuran.

4. The formulation of claim 3, wherein the active agent has the structure of formula (IV) 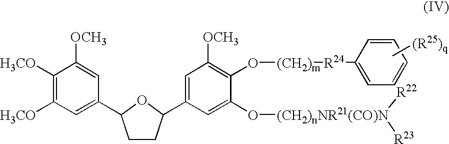
wherein n is 0 or 1, m is 2 or 3, q is 1, 2, 3 or 4, R²¹ is H or OH, R²² is H or OH, R²³ is lower alkyl, R²⁴ is S or SO₂, and R²⁵ is lower alkyl, lower alkoxy or halide.

5. The formulation of claim 1, wherein the active agent is (±) trans-2-[5-(N′-methyl-N-hydroxyureidylmethyl)-3-methoxy-4-p-chlorophenylthioethoxyphenyl]-5-(3,4,5-trimethoxy-phenyl)tetrahydrofuran.

6. The formulation of claim 1, wherein the active agent is (±) trans-2-[5-(N′-methyl-N′-hydroxyureidylmethyl)-3-methoxy-4-p-chlorophenylthiopropoxyphenyl]-5-(3,4,5-trimethoxy-phenyl)tetrahydrofuran.

7. The formulation of claim 1, wherein the active agent is (±) trans-2-[5-(N′-methyl-N′-hydroxyureidylmethyl)-3-methoxy-4-p-fluorophenylthioethoxyphenyl]-5-(3,4,5-trimethoxy-phenyl)tetrahydrofuran.

8. The formulation of claim 1, wherein the active agent is (±) trans-2-[5-(N′-methyl-N′-hydroxyureidylmethyl)-3-methoxy-4-p-fluorophenylthiopropoxyphenyl]-5-(3,4,5-trimethoxy-phenyl)tetrahydrofuran.

9. The formulation of claim 1, wherein the active agent used to prepare the formulation is in crystalline form.

10. The formulation of claim 4, wherein the active agent used to prepare the formulation is in crystalline form.

11. The formulation of claim 5, wherein the active agent used to prepare the formulation is in crystalline form.

12. The formulation of claim 1, wherein the enhancer composition comprise s an ester that is liquid at room temperature and has a molecular weight of less than about 250.

13. The formulation of claim 12, wherein the ester contains 3-18 carbon atoms and one to three ester functionalities.

14. The formulation of claim 1, wherein the enhancer composition comprises an ester selected from the group consisting of diethyl succinate, propylene carbonate, diisopropyl adipate, glyceryl triacetate, and combinations thereof.

15. The formulation of claim 14, wherein the enhancer composition comprises a mixture of two or more of diethyl succinate, propylene carbonate, diisopropyl adipate and glyceryl triacetate.

16. The formulation of claim 4, wherein the enhancer composition comprises an ester selected from the group of diethyl succinate, propylene carbonate, diisopropyl adipate, glyceryl triacetate, and combinations thereof.

17. The formulation of claim 16, wherein the enhancer composition comprises a mixture of two or more of diethyl succinate, propylene carbonate, diisopropyl adipate and glyceryl triacetate.

18. The formulation of claim 5, wherein the enhancer composition comprises an ester selected from the group consisting of diethyl succinate, propylene carbonate, diisopropyl adipate, glyceryl triacetate, and combinations thereof.

19. The formulation of claim 18, wherein the enhancer composition comprises a mixture of two or more of diethyl succinate, propylene carbonate, diisopropyl adipate and glyceryl triacetate.

20. The formulation of claim 1, comprising approximately 0.01 to 10.0 wt. % active agent.

21. The formulation of claim 1, comprising approximately 0.02 to 50 wt. % enhancer composition.

22. The formulation of claim 21, comprising approximately 0.02 to 20.0 wt. % enhancer composition.

23. A topical pharmaceutical formulation for treating inflammatory and/or immune disorders, comprising, in a topical carrier, a therapeutically effective amount of an active agent selected from the group consisting of 2,5-diaryl tetrahydrofurans, 2,5-diaryl tetrahydrothiophenes, 2,4-diaryl tetrahydrofurans, 2,4-diaryl tetrahydrothiophenes, 1,3-diaryl cyclopentanes, 2,4-diaryl pyrrolidines, and 2,5-diaryl pyrrolidines, wherein the active agent used to prepare the formulation is in crystalline form.

24. The formulation of claim 23, in the form of a cream.

25. The formulation of claim 23, in the form of a lotion.

26. The formulation of claim 23, in the form of a gel.

27. The formulation of claim 23, in the form of a solution.

28. The formulation of claim 23, in the form of an oil.

29. The formulation of claim 28, wherein the formulation comprises an alcohol.

30. The formulation of claim 28, wherein the formulation comprises a branched C₁₋₁₂ alkyl alcohol.

31. The formulation of claim 28, wherein the formulation comprises isopropyl alcohol.

32. The formulation of claim 28, wherein the formulation comprises isopropyl alcohol in an amount of 1 to about 30 weight percent, based on total weight of the formulation.

33. The formulation of claim 28, wherein the formulation comprises isopropyl alcohol in an amount of about 5 to about 15 weight percent, based on total weight of the formulation.

34. The formulation of claim 28, wherein the formulation comprises isopropyl alcohol in an amount of about 10 weight percent, based on total weight of the formulation.

35. The formulation of any one of claims 23-34, wherein the active agent is a 2,5-diaryl tetrahydrofuran, a 2,5-diaryl tetrahydrothiophene, a 2,5-diaryl pyrrolidine, or a 1,3-diaryl cyclopentane.

36. The formulation of claim 35, wherein the active agent is a 2,5-diaryl tetrahydrofuran.

37. The formulation of claim 35, wherein the active agent has the structure of formula (IV): 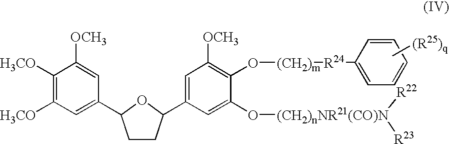
wherein n is 0 or 1, m is 2 or 3, q is 1, 2, 3 or 4, R²¹ is H or OH, R²² is H or OH, R²³ is lower alkyl, R²⁴ is S or SO₂, and R²⁵ is lower alkyl, lower alkoxy or halide.

38. The formulation of claim 23, wherein the active agent is (±) trans-2-[5-(N′-methyl-N′-hydroxyureidylmethyl)-3-methoxy-4-p-chlorophenylthioethoxyphenyl]-5-(3,4,5-trimethoxyphenyl)tetrahydrofuran.

39. The formulation of claim 23, wherein the active agent is (±) trans-2-[5-(N′-methyl-N′-hydroxyureidylmethyl)-3-methoxy-4-p-chlorophenylthiopropoxyphenyl]-5-(3,4,5-trimethoxyphenyl)tetrahydrofuran.

40. The formulation of claim 23, wherein the active agent is (±) trans-2-[5-(N′-methyl-N′-hydroxyureidylmethyl)-3-methoxy-4-p-fluorophenylthioethoxyphenyl]-5-(3,4,5-trimethoxyphenyl)tetrahydrofuran.

41. The formulation of claim 23, wherein the active agent is (±) trans-2-[5-(N′-methyl-N′-hydroxyureidylmethyl)-3-methoxy-4-p-fluorophenylthiopropoxyphenyl]-5-(3,4,5-trimethoxyphenyl)tetrahydrofuran.

42. The formulation of claim 23, further comprising an enhancer composition containing an ester that is liquid at room temperature and has a molecular weight of less than about 250.

43. The formulation of claim 42, wherein the ester contains 3-18 carbon atoms and one to three ester functionalities.

44. The formulation of claim 23, wherein the enhancer composition comprises an ester selected from the group consisting of diethyl succinate, propylene carbonate, diisopropyl adipate, glyceryl triacetate, and combinations thereof.

45. The formulation of claim 44, wherein the enhancer composition comprises a mixture of two or more of diethyl succinate, propylene carbonate, diisopropyl adipate and glyceryl triacetate.

46. The formulation of claim 37, wherein the enhancer composition comprises an ester selected from the group consisting of diethyl succinate, propylene carbonate, diisopropyl adipate, glyceryl triacetate, and combinations thereof.

47. The formulation of claim 46, wherein the enhancer composition comprises a mixture of two or more of diethyl succinate, propylene carbonate, diisopropyl adipate and glyceryl triacetate.

48. The formulation of claim 38, wherein the enhancer composition comprises an ester selected from the group consisting of diethyl succinate, propylene carbonate, diisopropyl adipate, glyceryl triacetate, and combinations thereof.

49. The formulation of claim 48, wherein the enhancer composition comprises a mixture of two or more of diethyl succinate, propylene carbonate, diisopropyl adipate and glyceryl triacetate.

50. A pharmaceutical formulation for rectal administration to treat inflammation of the gastrointestinal tract, comprising, in a suppository base, a therapeutically effective amount of an active agent selected from the group consisting of 2,5-diaryl tetrahydrofurans, 2,5-diaryl tetrahydrothiophenes, 2,4-diaryl tetrahydrofurans, 2,4-diaryl tetrahydrothiophenes, 1,3-diaryl cyclopentanes, 2,4-diaryl pyrrolidines, and 2,5-diaryl pyrrolidines.

51 A compound having the structure of formula (IV) 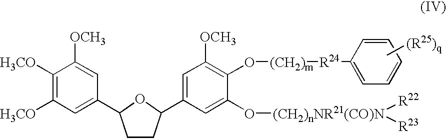
in crystalline form, wherein n is 0 or 1, m is 2 or 3, q is 1, 2, 3 or 4, R²¹ is H or OH, R²² is H or OH, R²³ is lower alkyl, R²⁴ is S or SO₂, and R²⁵ is lower alkyl, lower alkoxy or halide.

52. (±) Trans-2-[5-(N′-methyl-N′-hydroxyureidylmethyl)-3-methoxy-4-p-chlorophenylthioethoxyphenyl]-5-(3,4,5-trimethoxyphenyl)tetrahydrofuran, in crystalline form.

53. (±) Trans-2-[5-(N′-methyl-N′-hydroxyureidylmethyl)-3-methoxy-4-p-chlorophenylthiopropoxyphenyl]-5-(3,4,5-trimethoxyphenyl)tetrahydrofuran, in crystalline form.

54. (±) Trans-2-[5-(N′-methyl-N′-hydroxyureidylmethyl)-3-methoxy-4-p-fluorophenylthioethoxyphenyl]-5-(3,4,5-trimethoxyphenyl)tetrahydrofuran, in crystalline form.

55. (±) Trans-2-[5-(N′-methyl-N′-hydroxyureidylmethyl)-3-methoxy-4-p-fluorophenylthiopropoxyphenyl]-5-(3,4,5-trimethoxyphenyl)tetrahydrofuran, in crystalline form.

56. A method for treating an individual with an inflammatory and/or immune disorder, comprising applying the formulation of claim 1 to the skin, mucosal tissue or eye of the individual, within the context of a dosing regimen effective to provide the desired therapeutic result.

57. The method of claim 56, wherein the disorder is mediated by PAF or products of 5-lipoxygenase.

58. The method of claim 57, wherein the formulation is applied to the skin.

59. The method of claim 58, wherein the disorder is selected from the group consisting of psoriasis, contact dermatitis, atopic dermatitis, exfoliative dermatitis, seborrheic dermatitis, erythemas, discoid lupus erythematosus and dermatomyositis.

60. The method of claim 59, wherein the disorder is psoriasis.

61. The method of claim 59, wherein the disorder is atopic dermatitis.

62. A method for treating an individual with an inflammatory and/or immune disorder, comprising applying the formulation of claim 23 to the skin, mucosal tissue or eye of the individual, within the context of a dosing regimen effective to provide the desired therapeutic result.

63. The method of claim 62, wherein the disorder is mediated by PAF or products of 5-lipoxygenase.

64. The method of claim 63, wherein the formulation is applied to the skin.

65. The method of 64, wherein the disorder is selected from a group consisting of psoriasis, contact dermatitis, atopic dermatitis, exfoliative dermatitis, seborrheic dermatitis, erythemas, discoid lupus erythematosus and dermatomyositis.

66. The method of claim 65, wherein the disorder is psoriasis.

62. The method of claim 60, wherein the disorder is atopic dermatitis.

63. A method for treating an individual suffering from inflammation of the gastrointestinal tract, comprising rectally administering to the individual the formulation of claim 1.

64. A method for treating an individual suffering from inflammation of the gastrointestinal tract, comprising rectally administering to the individual the formulation of claim 23.

65. A method for treating an individual suffering from inflammation of the gastrointestinal tract, comprising rectally administering to the individual the formulation of claim 50.

66. A pharmaceutical formulation suitable for treating inflammatory and/or immune disorders, comprising, in carrier that comprises an oil and an alkyl alcohol, a therapeutically effective amount of an active agent selected from the group consisting of 2,5-diaryl tetrahydrofurans, 2,5-diaryl tetrahydrothiophenes, 2,4-diaryl tetrahydrofurans, 2,4-diaryl tetrahydrothiophenes, 1,3-diaryl cyclopentanes, 2,4-diaryl pyrrolidines, and 2,5-diaryl pyrrolidines.

67. The formulation of claim 66 wherein the active agent used to prepare the formulation is in crystalline form.

68. The formulation of claim 66 or 67 wherein the active agent is trans-2-[5-(N′-methyl-N′-hydroxyureidylmethyl)-3-methoxy-4-p-chlorophenylthioethoxyphenyl]-5-(3,4,5-trimethoxyphenyl)tetrahydrofuran.

69. The formulation of claim 66 wherein the formulation comprises a branched C₁₋₁₂ alkyl alcohol.

70. The formulation of claim 66 wherein the formulation comprises isopropyl alcohol.

71. The formulation of claim 66 wherein the formulation comprises isopropyl alcohol in an amount of 1 to about 30 weight percent, based on total weight of the formulation.

72. The formulation of claim 66 wherein the formulation comprises isopropyl alcohol in an amount of about 5 to about 15 weight percent, based on total weight of the formulation.

73. The formulation of claim 66 wherein the formulation comprises isopropyl alcohol in an amount of about 10 weight percent, based on total weight of the formulation.

74. A method for treating an individual with an inflammatory and/or immune disorder, comprising applying the formulation of claim 66 to the skin, mucosal tissue or eye of the individual, within the context of a dosing regimen effective to provide the desired therapeutic result.

75. A composition comprising an active agent selected from the group consisting of 2,5-diaryl tetrahydrofurans, 2,5-diaryl tetrahydro-thiophenes, 2,4-diaryl tetrahydrofurans, 2,4-diaryl tetrahydrothiophenes, 1,3-diaryl cyclopentanes, 2,4-diaryl pyrrolidines, and 2,5-di aryl pyrrolidines, and an effective amount of an alcoholic enhancer composition.

76. The composition of claim 75, wherein the alcoholic enhancing composition is an alcohol.

77. The composition of claim 76, wherein the alcohol is a C3 alcohol.

78. The composition of claim 77, wherein the C3 alcohol is isopropyl alcohol.
